SEQUENCE LISTING (18 sequences)

SEQ ID NO: 1 - protein, 504 aa, Artificial Sequence
  Brazil-ZKV2015
IRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGTQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLMWLGLNTKNGSISLMCLALGGVLIFLSTAVSA

SEQ ID NO: 2 - protein, 504 aa, Artificial Sequence
  SPH2015-Brazil
IRCIGVSNRDFVEGMSGGTWVDIVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLMWLGLNTKNGSISLMCLALGGVLIFLSTAVSA

SEQ ID NO: 3 - protein, 504 aa, Artificial Sequence
  FSS13025-Cambodia
IRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLVWLGLNTKNGSISLMCLALGGVLIFLSTAVSA

SEQ ID NO: 4 - protein, 498 aa, Artificial Sequence
  IbH_30656-Nigeria
IRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFTCSKKMTGKSIQPENLEYRIMLSVHGSQHSGMIVNDENRAKVEVTPNSPRAEATLGGFGSLGLDCEPRTGLDFSDLYYLTMNNKHWLVHKEWFHDIPLPWHSGADTETPHWNNKEALVEFKDAHAKRQTVVVLGSQEGAVHTALAGALEAEMDGAKGRLSSGHLKCRLKMDKLRLKGVSYSLCTAAFTFTKVPAETLHGTVTVEVQYAGRDGPCKVPAQMAVDMQTLTPVGRLITANPVITESTENSKMMLELDPPFGDSYIVIGVGDKKITHHWHRSGSIIGKAFEATVRGAKRMAVLGDTAWDFGSVGGVFNSLGKGIHQIFGAAFKSLFGGMSWFSQILIGTLLVWLGLNTKNGSISLTCLALGGVMIFLSTAVSA

SEQ ID NO: 5 - protein, 504 aa, Artificial Sequence
  H/PF/2013-Polynesia
IRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLMWLGLNTKNGSISLMCLALGGVLIFLSTAVSA

SEQ ID NO: 6 - protein, 504 aa, Artificial Sequence; identical to SEQ ID NO: 5
  PRVABC59-Puerto Rico

SEQ ID NO: 7 - protein, 504 aa, Artificial Sequence; identical to SEQ ID NO: 5
  PLCal_ZV-Thailand

SEQ ID NO: 8 - protein, 504 aa, Artificial Sequence
  MR 766-Uganda
IRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFTCSKKMTGKSIQPENLEYRIMLSVHGSQHSGMTVNDIGYETDENRAKVEVTPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGKLFSGHLKCRLKMDKLRLKGVSYSLCTAAFTFTKVPAETLHGTVTVEVQYAGTDGPCKIPVQMAVDMQTLTPVGRLITANPVITESTENSKMMLELDPPFGDSYIVIGVGDKKITHHWHRSGSTIGKAFEATVRGAKRMAVLGDTAWDFGSVGGVFNSLGKGIHQIFGAAFKSLFGGMSWFSQILIGTLLVWLGLNTKNGSISLTCLALGGVMIFLSTAVSA

SEQ ID NO: 9 - protein, 352 aa, Artificial Sequence
  Brazil-ZKV2015
DVGCSVDFSKKETRCGTGVFVYNDVEAWRDRYKYHPDSPRRLAAAVKQAWEDGICGISSVSRMENIMWRSVEGELNAILEENGVQLTVVVGSVKNPMWRGPQRLPVPVNELPHGWKAWGKSYFVRAAKTNNSFVVDGDTLKECPLKHRAWNSFLVEDHGFGVFHTSVWLKVREDYSLECDPAVIGTAVKGKEAVHSDLGYWIESEKNDTWRLKRAHLIEMKTCEWPKSHTLWTDGIEESDLIIPKSLAGPLSHHNTREGYRTQMKGPWHSEELEIRFEECPGTKVHVEETCGTRGPSLRSTTASGRVIEEWCCRECTMPPLSFRAKDGCWYGMEIRPRKEPESNLVRSMVTA

SEQ ID NO: 10 - protein, 352 aa, Artificial Sequence
  SPH2015-Brazil
DVGCSVDFSKKETRCGTGVFVYNDVEAWRDRYKYHPDSPRRLAAAVKQAWEDGICGISSVSRMENIMWRSVEGELNAILEENGVQLTVVVGSVKNPMWRGPQRLPVPVNELPHGWKAWGKSHFVRAAKTNNSFVVDGDTLKECPLKHRAWNSFLVEDHGFGVFHTSVWLKVREDYSLECDPAVIGTAVKGKEAVHSDLGYWIESEKNDTWRLKRAHLIEMKTCEWPKSHTLWTDGIEESDLIIPKSLAGPLSHHNTREGYRTQMKGPWHSEELEIRFEECPGTKVHVEETCGTRGPSLRSTTASGRVIEEWCCRECTMPPLSFRAKDGCWYGMEIRPRKEPESNLVRSMVTA

SEQ ID NO: 11 - protein, 352 aa, Artificial Sequence
  FSS13025-Cambodia
DVGCSVDFSKKETRCGTGVFVYNDVEAWRDRYKYHPDSPRRLAAAVKQAWEDGICGISSVSRMENIMWRSVEGELNAILEENGVQLTVVVGSVKNPMWRGPQRLPVPVNELPHGWKAWGKSYFVRAAKTNNSFVVDGDTLKECPLKHRAWNSFLVEDHGFGVFHTSVWLKVREDYSLECDPAVIGTAAKGKEAVHSDLGYWIESEKNDTWRLKRAHLIEMKTCEWPKSHTLWTDGIEESDLIIPKSLAGPLSHHNTREGYRTQMKGPWHSEELEIRFEECPGTKVHVEETCGTRGPSLRSTTASGRVIEEWCCRECTMPPLSFRAKDGCWYGMEIRPRKEPESNLVRSMVTA

SEQ ID NO: 12 - protein, 352 aa, Artificial Sequence
  IbH_30656-Nigeria
DVGCSVDFSKKETRCGTGVFIYNDVEAWRDRYRYHPDSPRRLAAAVKQAWEEGICGISSVSRMENIMWKSVEGELNAILEENGVQLTVVVGSVKNPMWRGPRRLPVPVNELPHGWKAWGKSYFVRAAKTNNSFVVDGDTLKECPLKHRAWNSFLVEDHGFGVFHTSVWLKVREDYSLECDPAVIGTAVKGKEAAHSDLGYWIESEKNDTWRLKRAHLIEMKTCEWPKSHTLWTDGVEESDLIIPKSLAGPLSHHNTREGYRTQVKGPWHSEELEIRFEECPGTKVHVEETCGTRGPSLRSTTASGRVIEEWCCRECTMPPLSFRAKDGCWYGMEIRPRKEPESNLVRSMVTA

SEQ ID NO: 13 - protein, 352 aa, Artificial Sequence; identical to SEQ ID NO: 9
  H/PF/2013-Polynesia

SEQ ID NO: 14 - protein, 352 aa, Artificial Sequence; identical to SEQ ID NO: 9
  PRVABC59-Puerto Rico

SEQ ID NO: 15 - protein, 352 aa, Artificial Sequence; identical to SEQ ID NO: 9
  PLCal_ZV-Thailand

SEQ ID NO: 16 - protein, 352 aa, Artificial Sequence
  MR 766-Uganda
DVGCSVDFSKKETRCGTGVFIYNDVEAWRDRYKYHPDSPRRLAAAVKQAWEEGICGISSVSRMENIMWKSVEGELNAILEENGVQLTVVVGSVKNPMWRGPQRLPVPVNELPHGWKAWGKSYFVRAAKTNNSFVVDGDTLKECPLEHRAWNSFLVEDHGFGVFHTSVWLKVREDYSLECDPAVIGTAVKGREAAHSDLGYWIESEKNDTWRLKRAHLIEMKTCEWPKSHTLWTDGVEESDLIIPKSLAGPLSHHNTREGYRTQVKGPWHSEELEIRFEECPGTKVYVEETCGTRGPSLRSTTASGRVIEEWCCRECTMPPLSFRAKDGCWYGMEIRPRKEPESNLVRSMVTA

SEQ ID NO: 17 - protein, 504 aa, Artificial Sequence; identical to SEQ ID NO: 5
  Envelope Protein (Consensus Sequence)

SEQ ID NO: 18 - protein, 352 aa, Artificial Sequence; identical to SEQ ID NO: 9
  NS1 Protein (Consensus Sequence)

(19) United States
(12) Patent Application Publication
(10) Pub. No.: US 2021/0228717 A1
(43) Pub. Date: Jul. 29, 2021

(54) MULTIANALYTE IMMUNOASSAY AND USES THEREOF

(71) Applicant: Emergent Biosolutions Canada Inc., Winnipeg (CA)

(72) Inventors: Russell PRONYK, Winnipeg (CA); Samantha ERICHSEN, Winnipeg (CA)

(21) Appl. No.: 17/051,019

(22) PCT Filed: Apr. 26, 2019
(86) PCT No.: PCT/IB2019/053463

§ 371 Date: Oct. 27, 2020

Related U.S. Application Data
(60) Provisional application No. 62/663,972, filed on Apr. 27, 2018.

(51) Int. Cl. A61K 39/42; C07K 14/005; G01N 33/569; G01N 33/68

(52) U.S. Cl. CPC A61K 39/42; C07K 14/005; C12N 2770/24122; G01N 33/6854; G01N 2333/185; G01N 33/56983

(57) ABSTRACT

The present disclosure is directed to compositions (e.g., multianalyte immunoassays) for detecting multiple Zika virus antibodies in a biological sample. Also disclosed herein are methods for identifying suitable donors for preparing a Zika virus hyperimmune composition. Methods for selectively detecting Zika virus antibodies in biological samples that may contain other flavivirus antibodies are also provided. Methods of treating, preventing, or reducing the risk of a Zika virus infection are also provided.

### CROSS-REFERENCE TO RELATED APPLICATIONS

This application claims priority to U.S. Provisional Application 62/663,972, filed Apr. 27, 2018, which is herein incorporated by reference in its entirety.

### REFERENCE TO SEQUENCE LISTING SUBMITTED ELECTRONICALLY VIA EFS-WEB

The content of the electronically submitted sequence listing in ASCII text file (Name: 2479.199PC01_ST25.txt; Size: 67,758 bytes; and Date of Creation: Apr. 26, 2019) filed with the application is herein incorporated by reference in its entirety.

### BACKGROUND

Zika virus (Zika) belongs to the genus Flavivirus within the family Flaviviridae. Many flaviviruses are significant human pathogens, including Zika, yellow fever, dengue virus, Japanese encephalitis, West Nile virus, and tick-borne encephalitis virus. Wong S J, et al., EBioMedicine 16:136-140 (2017). Zika is predominantly transmitted by mosquitoes but can also be transmitted through maternofetal route, sexual intercourse, blood transfusion, and organ transplantation. Musso, D, et al., Clinical Microbiology Review, 29(3):487-524 (2016). While the majority of Zika infections are asymptomatic, symptoms of infection can include: headaches, fever, lethargy, rash, conjunctivitis, myalgia, and arthralgia. In severe cases, infection can result in neurotropic Guillain-Barré syndrome and congenital microcephaly. Weaver, S C, et al., Antiviral Research, 130:69-80 (2016).

Like other Flavivirus, the Zika genome consists of a single-strain, positive-sense RNA of approximately 11,000 nucleotides. It contains a 5′ untranslated region (UTR), and open-reading frame (ORF), and a 3′ UTR. The single ORF encodes a long polyprotein which is processed into ten viral proteins: including three structural proteins (capsid (C), precursor membrane (prM), and envelope (E)) and seven non-structural proteins (NS1, NS2A, NS2B, NS3, NS4A, NS4B, and NS5). Lindenbach, B D, et al., 2013. Flaviviridae. In: Knipe, D. M., Howley, P. M. (Eds.), Fields Virology, 6th vol. 1. Lippincott William & Wilkins, Philadelphia, pp. 712-746.

Recent Zika virus epidemic in the Americas (e.g., French Polynesia in 2013 and in Brazil, Colombia, and Cape Verde in 2015) highlighted the potential severity of the virus. The exact global distribution of the virus worldwide is still not yet well understood. Mayer, S V, et al., Acta Prop 166:155-163 (2017).

Traditional assays for detecting Zika virus are ELISA-based and rely on specificity to a single Zika antigen (e.g., NS1). Bosch, I, et al., Science Translational Medicine 9:eaan1589 (2017). However, different proteins of the different flaviviruses are highly conserved across different species, and an individual infected against a non-Zika Flavivirus can have antibodies that can bind to Zika antigens due to cross-reactivity. See Gyurech, D, et al., Swiss Medicine Weekly 146:w14296 (2016). Therefore, to reduce the likelihood of possible misdiagnosis (e.g., false positive), positive test results have to be confirmed by virus neutralization tests, which are time consuming and often require BSL-3 laboratories. Moreover, these cross-reactive antibodies are known to be poor neutralizing antibodies and can contribute to Antibody Dependent Enhancement (ADE).

There are no reported multianalyte tests available for reliably distinguishing between Zika virus antibodies and other flavivirus antibodies. Furthermore, there are currently no approved treatments or vaccines available for Zika virus infection. See Chen, L H and Hamer D H, Annuals of Internal Medicine, 164(9):613-615 (2016).

### BRIEF SUMMARY

One aspect of the present disclosure is directed to an in vitro method for identifying a donor for use in preparing a Zika virus hyperimmune composition. In some embodiments, the method comprises determining a level of an antibody against a Zika Non-Structural protein 1 (anti-NS1 antibody) and a level of an antibody against a Zika Envelope protein (anti-E-protein antibody) in a biological sample from a potential donor; wherein the potential donor is the donor for use in preparing a Zika virus hyperimmune composition if (i) both the anti-NS1 antibody and the anti-E-protein antibody are present in the biological sample; and (ii) the ratio of the level of the anti-NS1 antibody to the level of the anti-E-protein antibody is greater than about 0.6.

An additional aspect of the present disclosure is directed to a method of preparing a Zika virus hyperimmune composition. In some embodiments, the method comprises (a) identifying the donor for use in preparing the Zika virus hyperimmune composition according to the methods disclosed herein. The method may further include (b) collecting plasma and/or serum from the donor. The method may further include (c) pooling the collected plasma and/or serum. The method may further include (d) processing the pooled plasma and/or serum.

A further aspect of the present disclosure is directed to a method for differentiating between Zika virus antibodies and Non-Zika flavivirus antibodies in a biological sample from a subject. In some embodiments, the method comprises (a) detecting the presence or absence of antibodies against a Zika Non-Structural protein 1 (anti-NS1 antibody) and the presence or absence of antibodies against a Zika Envelope protein (anti-E-protein antibody) in a biological sample, wherein the presence of both the anti-NS1 antibodies and the anti-E-protein antibodies indicates that the sample is a flavivirus-positive biological sample. The method may further include (b) determining a level of the anti-NS1 antibody and a level of the anti-E-protein antibody in the flavivirus positive biological sample; wherein the flavivirus positive biological sample is from a subject with Zika-virus antibodies if the level of the anti-NS1 antibody is greater than the level of the anti-E-protein antibody.

An additional aspect of the present disclosure is directed to an in vitro method for detecting two target antibodies present in a biological sample. In some embodiments, the method comprises (a) contacting the biological sample with a first solid support bound to a first ligand, which binds to a variable region of a first target antibody, wherein the first target antibody is a Zika Non-structural Protein 1 (NS1) antibody (anti-NS1 antibody). The method may further include (b) contacting the biological sample with a second solid support bound to a second ligand, which binds to a variable region of a second target antibody, wherein the second target antibody is a Zika Envelope-protein (E-protein) antibody (anti-E-protein antibody). The method may further include (c) detecting the presence or absence of the two target antibodies by detecting the binding or lack of binding of the first target antibody and the second target antibody to the first ligand and second ligand, respectively.

Another aspect of the present disclosure is directed to a solid support. In some embodiments, the solid support comprises a first ligand and a second ligand, wherein the first ligand binds to a variable region of a Zika Non-structural Protein 1 (NS1) antibody (anti-NS1 antibody) and the second ligand binds to a variable region of a Zika Envelope-protein antibody (E-protein) (anti-E-protein antibody), wherein the first ligand and the second ligand are immobilized on the solid support.

An additional aspect of the present disclosure is directed to a solution. In some embodiments, the solution comprises a first ligand, a second ligand, and a plurality of beads, wherein the first ligand binds to a variable region of a Zika Non-structural Protein 1 (NS1) antibody (anti-NS1 antibody) and the second ligand binds to a variable region of a Zika Envelope-protein (E-protein) antibody (anti-E-protein antibody), wherein the plurality of beads attach to complexes formed by the first ligand bound to the variable region of the anti-NS1 antibody and/or to complexes formed by the second ligand bound to the variable region of the anti-E-protein antibody.

Another aspect of the present disclosure is directed to a kit. In some embodiments, the kit comprises a solid support or a solution. In some embodiments, the solid support comprises a first ligand and a second ligand, wherein the first ligand binds to a variable region of a Zika Non-structural Protein 1 (NS1) antibody (anti-NS1 antibody) and the second ligand binds to a variable region of a Zika Envelope-protein antibody (E-protein) (anti-E-protein antibody), wherein the first ligand and the second ligand are immobilized on the solid support. In some embodiments, the solution comprises a first ligand, a second ligand, and a plurality of beads, wherein the first ligand binds to a variable region of a Zika Non-structural Protein 1 (NS1) antibody (anti-NS1 antibody) and the second ligand binds to a variable region of a Zika Envelope-protein (E-protein) antibody (anti-E-protein antibody), wherein the plurality of beads attach to complexes formed by the first ligand bound to the variable region of the anti-NS1 antibody and/or to complexes formed by the second ligand bound to the variable region of the anti-E-protein antibody.

An additional aspect of the present disclosure is directed to preparing a Zika hyperimmune composition.

Another aspect of the present disclosure is directed to a method of treating, preventing, or reducing the risk of a Zika virus infection in a subject. In some embodiments, the method comprises administering a Zika virus hyperimmune composition to the subject.

Also provided are Zika virus hyperimmune compositions prepared according to the method disclosed herein. In some embodiments, the hyperimmune compositions can be used for treating, preventing, or reducing the risk of a Zika virus infection in a subject.

Other aspects and iterations of the present disclosure are described in more detail below.

### BRIEF DESCRIPTION OF THE DRAWINGS

FIG. 1 provides a sequence alignment of exemplary Zika virus Envelope proteins. The E proteins from the following strains are shown: (i) Brazil_ZKV2015 (SEQ ID NO: 1); (ii) SPH2015-Brazil (SEQ ID NO: 2); (iii) FSS13025-Cambodia (SEQ ID NO: 3); (iv) IbH_30656-Nigeria (SEQ ID NO: 4); (v) H/PF/2013-Polynesia (SEQ ID NO: 5); (vi) PRVABC59-Puerto Rico (SEQ ID NO: 6); (vii) PLCal_ZV-Thailand (SEQ ID NO: 7); and (viii) MR 766-Uganda (SEQ ID NO: 8). The sequence shown at the very top is the consensus sequence (SEQ ID NO: 17) generated based on the sequence alignment of all the Envelope protein sequences.

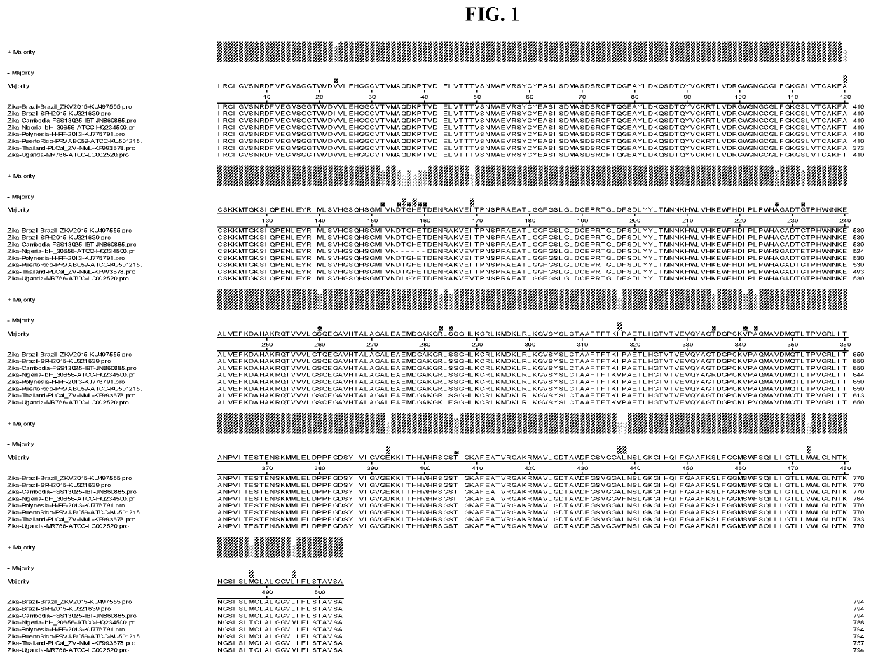

FIG. 2 provides a sequence alignment of exemplary Zika virus NS1 proteins. The NS1 proteins from the following strains are shown: (i) H/PF/2013-Polynesia (SEQ ID NO: 13); (ii) Brazil_ZKV2015 (SEQ ID NO: 9); (iii) SPH2015-Brazil (SEQ ID NO: 10); (iv) FSS13025-Cambodia (SEQ ID NO: 11); (v) MR 766-Uganda (SEQ ID NO: 16); (vi) IbH_30656-Nigeria (SEQ ID NO: 12); (vii) PRVABC59-Puerto Rico (SEQ ID NO: 14); and (viii) PLCal_ZV-Thailand (SEQ ID NO: 15). The sequence shown at the very top is the consensus sequence (SEQ ID NO: 18) generated based on the sequence alignment of all the NS1 protein sequences.

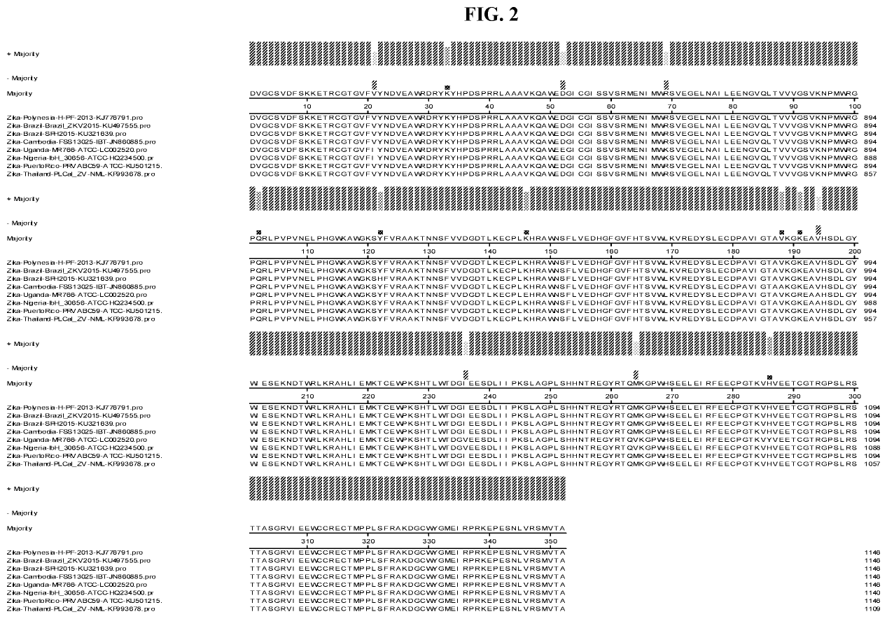

FIG. 3 shows a comparison of the median fluorescent intensity (MFI) of the Zika anti-NS1 antibody and the Zika anti-E-protein antibody in various samples. The samples tested include: (i) plasma from four different Dengue infected individuals (DENV1, DENV2, DENV3, and DENV4), (ii) plasma from nine different Zika infected individuals (Zika 1-9), (iii) plasma from Yellow fever vaccinated individuals (YF Vac.), and (iv) plasma from normal (i.e., uninfected) individuals (Normal Plasma). The Normal Plasma result shown on the graph is the mean result of 10 individuals. The yellow fever vaccinated result is the mean result of 7 individuals. The bars shown correspond to beads coupled to different antigens: Zika lysate, Zika virus NS1 protein, Zika Envelope protein, and Type 2 Dengue antigen (1^{st}, 2^{nd}, 3^{rd}, and 4^{th}, respectively, from left to right).

FIG. 4 shows a ratio of the NS1 median fluorescent intensity (MFI) signal to the E-protein MFI signal for plasma samples from Dengue infected individuals (DENV1, DENV2, DENV3, DENV4, and DENV5) and plasma samples from Zika virus infected individuals (Zika 1-9).

### DETAILED DESCRIPTION

There is a need for improved methods for identifying plasma and/or serum donors suitable for use in preparing Zika virus hyperimmune compositions and more accurate diagnostic tools for distinguishing between a subject or subjects who previously had a Zika infection and a subject or subjects who previously had a non-Zika flavivirus infection.

The present disclosure provides a multianalyte immunoassay, which can measure the levels of antibodies that bind Zika envelope protein (E-protein) and antibodies that bind Zika non-structural protein 1 (NS1) in a single assay. Use of this assay can more accurately identify suitable plasma and/or serum donors (e.g., an individual or pool of individuals who were previously infected with the Zika virus) for preparing Zika virus hyperimmune compositions. The multianalyte immunoassay of the present disclosure is also useful in differentiating biological samples from subjects with different flavivirus antibodies. For example, in some embodiments, the multianalyte immunoassay can be used to differentiate between a flavivirus-positive biological sample from a subject or subjects with Zika virus antibodies (e.g., a subject previously infected with a Zika virus) or from a subject or subjects with non-Zika flavivirus (e.g., Dengue) antibodies (e.g., a subject previously infected with a non-Zika flavivirus). The multianalyte immunoassay of the present disclosure can also be used to differentiate a flavivirus-positive biological sample and a biological sample that is positive for a related (e.g., having a high degree of homology with flaviviruses) family of viruses, e.g., alphaviruses (e.g., Chikungunya virus).

The present disclosure further provides methods of making hyperimmune compositions using the plasma and/or serum from donors identified by the disclosed methods. These Zika virus hyperimmune compositions, which can be used to treat, prevent, or reduce the risk of a Zika virus infection, are also disclosed.

### I. METHODS FOR IDENTIFYING HYPERIMMUNE DONORS

Certain aspects of the present disclosure are directed to methods for identifying a suitable donor for use in preparing a Zika virus hyperimmune composition.

Screening for anti-Zika virus-specific antibodies to make a human hyperimmune composition poses challenges due to epitope conservation between flaviviruses (e.g., Dengue, Japanese Encephalitis, Yellow Fever, West Nile, and Zika virus). The methods of the current application overcome this challenge. In some embodiments, the method for identifying a donor or plurality of donors (e.g., a plasma or serum donor or pool of donors) for use in preparing a Zika virus hyperimmune composition comprises: determining a level of an antibody against a Zika Non-Structural protein 1 (anti-NS1 antibody) and a level of an antibody against a Zika Envelope protein (anti-E-protein antibody) in a biological sample (e.g., a plasma or serum sample or pooled plasma or serum samples) from a potential donor or a plurality of potential donors; wherein the potential donor is a donor suitable for use in preparing a Zika virus-specific hyperimmune composition if (i) both the anti-Zika-NS1 antibody and the anti-Zika-E-protein antibody are present in the biological sample; and (ii) the ratio of the level of the anti-Zika-NS1 antibody to the level of the anti-Zika-E-protein antibody is greater than a borderline ratio. As used herein, “borderline ratio” refers to the ratio of the level of a first antibody (e.g., the anti-NS1 antibody) to the level of a second antibody (e.g., the anti-E-protein antibody) observed in a borderline control sample. As used herein, “borderline control sample” refers to a biological sample (e.g., pooled plasma or serum) from a donor positive for a related virus (e.g., Dengue positive donors). In some embodiments, the level of the first antibody in a potential donor is greater than the level of the second antibody in a potential donor (e.g., ratio of the anti-NS1 antibody level to the anti-E-protein antibody level is greater than 1).

In certain aspects, there can be two criteria/cut-offs to consider for any biological sample, one for specificity, and one for titer.

A “% of Positive criteria” can be used for determining if there is enough antibody (e.g., in a biological sample) to be detected in a neutralizing assay and thereby meaningfully contribute to a hyperimmune product. A low/mid/high cut off can be established for production planning to rank/compare donors. In some embodiments, subjects with low levels of unknown non-specific binding can be excluded.

According to certain aspects of the present disclosure, the level of the anti-NS1 antibody relative to that of the positive control sample (e.g., pooled Zika virus positive plasma samples) can be determined with the following formula: [(background subtracted anti-NS1 antibody level in the test sample)/(background subtracted anti-NS1 antibody level in the control sample)]×100. The background subtracted antibody level can be determined as follows: (antibody level in a sample)−(antibody level in the blank sample), wherein the blank sample includes no addition of any plasma (e.g., PBS control). As used herein, the term “positive control sample” refers to a biological sample that has been confirmed (e.g., through a neutralization assay) to contain specific antibodies (e.g., plasma from an individual or pool of individuals who are positive for Zika virus specific antibodies).

In some embodiments, the level of the anti-NS1 antibody in the test sample of a subject (e.g., plasma or serum from a potential donor or pool of donors) relative to the level of anti-NS1 antibody in a positive control sample can be used to determine the relative titer of anti-Zika virus-specific antibodies present in the subject (e.g., potential donor or pool of donors). In some embodiments, a subject may be determined to have an elevated level of anti-Zika virus-specific antibodies (e.g., compared to a subject who has never been infected with a Zika virus or exposed to one or more Zika virus antigens (e.g., the E-protein, and/or the NS1 protein, and/or whole inactivated or attenuated Zika virus)) where the level of the anti-NS1 antibody in the subject's test sample relative to the level of the positive control sample (e.g., plasma obtained from or plasma pooled from one or more individuals known to have been infected by Zika virus or exposed to one or more Zika virus antigens (e.g., the E-protein, and/or the NS1 protein, and/or whole inactivated or attenuated Zika virus)) may be greater than about 5%, greater than about 10%, greater than about 20%, greater than about 30%, greater than about 40%, greater than about 50%, greater than about 60%, greater than about 70%, greater than about 80%, or more. In some embodiments, the level of the anti-NS1 antibody in the subject's test sample relative to the level of the positive control sample may be between about 20% to about 40%, where the subject has low titer of Zika virus-specific antibodies. In some embodiments, the level of the anti-NS1 antibody in the subject's test sample relative to the level of the positive control sample may be between about 40% to about 70%, where the subject has medium titer of Zika virus-specific antibodies. In some embodiments, the level of the anti-NS1 antibody in the subject's test sample relative to the level of the positive control sample may be greater than about 70%, where the subject has high titer of Zika virus-specific antibodies. As disclosed herein, an individual or pool of individuals who are suitable plasma or serum donors for preparing a Zika virus hyperimmune composition have an elevated level of anti-Zika virus-specific antibodies. In some embodiments, a suitable plasma or serum donor may have a low, medium, or high antibody titer against the Zika virus. In some embodiments, the cutoff for the level of anti-NS1 antibody present in the subject's test sample used to determine whether an individual or pool of individuals may be considered to have low, medium, or high antibody titer against the Zika virus can change based on the level of the anti-NS1 antibody present in the positive control sample.

In some embodiments, the level of the anti-NS1 antibody in the biological sample may be at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, or at least 80% of a level of the anti-NS1 antibody in a positive control sample, e.g., obtained from one or more individuals previously infected with Zika virus.

In some embodiments, the level of the anti-NS1 antibody in the biological sample may be at least 20% of the level of the anti-NS1 antibody in a positive control sample.

A ratio criteria can be used for specificity. Thus, in some embodiments, the method for identifying a plasma or serum donor or pool of donors suitable for use in preparing a hyperimmune composition (e.g., for Zika virus) may comprise determining a ratio of the level of a first antibody (e.g., an anti-NS1 antibody) to the level of a second antibody (e.g., an anti-E-protein antibody) in the biological sample obtained from a potential donor or pool of donors, wherein the ratio is greater than about 0.1, greater than about 0.2, greater than about 0.3, greater than about 0.4, greater than about 0.5, greater than about 0.6, greater than about 0.625, greater than about 0.65, greater than about 0.675, greater than about 0.7, greater than about 0.75, greater than about 0.8, greater than about 0.85, greater than about 0.9, greater than about 0.95, greater than about 1.0, greater than about 1.5, greater than about 2.0, greater than about 2.5, greater than about 3.0, greater than about 3.5, greater than about 4.0, greater than about 4.5, or greater than about 5.0. In certain embodiments, the ratio of the level of the anti-NS1 antibody to the level of the anti-E-protein antibody in the biological sample obtained from a potential donor or pool of donors may be greater than about 0.6. In other embodiments, the ratio may be greater than about 1.0. The ratio of the anti-NS1 antibody to the anti-E-protein antibody may be calculated using the following formula: (background subtracted anti-NS1 antibody level in the test sample)/(background subtracted anti-E-protein antibody level in the test sample).

In some embodiments, a ratio (NS1/E-protein) greater than 0.5, greater than 0.55, greater than 0.6, greater than 0.625, greater than 0.65, greater than 0.675, greater than 0.7, greater than 0.725, greater than 0.75, greater than 0.775, greater than 0.8, greater than 0.825, greater than 0.85, greater than 0.875, greater than 0.9, greater than 0.925, or greater than 0.95 may mean the measured antibody response of the donor or donor pool was likely due to exposure to Zika virus and not a non-Zika flavivirus. In some embodiments, a donor or the pooled donor sample (e.g., plasma) has a ratio (NS1/E-protein) greater than 0.5, greater than 0.525, greater than 0.55, greater than 0.575, greater than 0.6, greater than 0.625, greater than 0.65, greater than 0.675, greater than 0.7, greater than 0.725, greater than 0.75, greater than 0.775, greater than 0.8, greater than 0.825, greater than 0.85, greater than 0.875, greater than 0.9, greater than 0.925, or greater than 0.95.

In some embodiments, a donor for use in preparing a Zika virus hyperimmune composition was not previously infected with a non-Zika flavivirus. In some embodiments, a donor for use in preparing a Zika virus hyperimmune composition was previously infected with a non-Zika flavivirus.

Upon identification of one or more donors meeting (i) the ratio criteria or (ii) both the ratio and 20% of the level of the anti-NS1 antibody in a positive control sample criteria, plasma and/or serum from the one or more donors can be collected for preparing the Zika virus hyperimmune composition. In some embodiments, the method may further comprise preparing immunoglobulin from the plasma and/or serum collected from the one or more donors. In some embodiments, the method may further comprise pooling (e.g., from the same or different donors), the collected plasma, collected serum, or prepared immunoglobulin for preparing the Zika virus hyperimmune composition. In some embodiments, the method may further comprise processing the pooled plasma, serum, or immunoglobulin for preparing the Zika virus hyperimmune composition. As used herein, the term “processing” comprises antibody (e.g., IgG) purification, viral inactivation and/or removal, microbial inactivation and/or removal, or combinations thereof. Antibody purification may be done by any method known in the art (e.g., affinity chromatography or using methods described in Chapter 14, Price et al., Production of Plasma Proteins for Therapeutic Use by Neil Goss). Viral and microbial inactivation and/or removal can also be done by any method known in the art (e.g., pasteurization, solvent/detergent, low pH solutions, precipitation, chromatography, or nanofiltration).

The hyperimmune composition may comprise a blood product, e.g., plasma, serum, immunoglobulins, or any combination thereof, from a donor(s) or pool of donor samples. In certain embodiments, the blood product may be from a donor(s) or pool of donor samples identified by the methods of the application and used to prepare a Zika virus hyperimmune composition.

In some embodiments, methods for identifying donors relate to detection and measuring a particular subset of antibodies (i.e., antibodies that bind to the Zika NS1 and Envelope proteins), which can serve as an indicator of a previous Zika virus infection, including assays for identification of suitable donors for preparing a Zika virus hyperimmune compositions, e.g., for determining if a flavivirus-positive biological sample is from a subject or subjects previously infected with a Zika virus or from a subject or subjects previously infected with a non-Zika flavivirus and methods using plasma and/or serum derived from hyperimmune donors. Certain compositions (e.g., multianalyte assays and solid supports) are used for detecting the multiple antibodies in a biological sample.

A biological sample from a potential donor may be contacted with a first ligand and a second ligand, wherein the first ligand binds to a variable region of an anti-NS1 antibody and wherein the second ligand binds to a variable region of an anti-E-protein antibody. In some embodiments, the first ligand may be bound to a first solid support and the second ligand is bound to a second solid support. In certain embodiments, the first solid support may be contacted with a first detectable label and the second solid support is contacted with a second detectable label, wherein the first detectable label binds to a constant region of the anti-NS1 antibody and wherein the second detectable label binds to a constant region of the anti-E-protein antibody.

In some embodiments, the first ligand may comprise a Zika NS1 polypeptide or antibody binding fragment thereof. In certain embodiments, the Zika NS1 polypeptide or antibody binding fragment thereof may comprise a sequence having at least 80%, at least 85%, at least 90%, at least 91%, at least 92%, at least 93%, at least 94%, at least 95%, at least 96%, at least 97%, at least 98%, at least 99%, or 100% amino acid sequence identity to a sequence selected from SEQ ID NOs: 9-16 or 18.

In some embodiments, the second ligand may comprise a Zika E-protein polypeptide or antibody binding fragment thereof. In certain embodiments, the Zika E-protein polypeptide or antibody binding fragment thereof may comprise a sequence having at least 80%, at least 85%, at least 90%, at least 91%, at least 92%, at least 93%, at least 94%, at least 95%, at least 96%, at least 97%, at least 98%, at least 99%, or 100% amino acid sequence identity to a sequence selected from SEQ ID NOs: 1-8 or 17.

In some embodiments, the first ligand and the second ligand may be in a ratio of about 1:0.1 to about 1:2, about 1:0.1 to about 1:1.5, about 1:0.5 to about 1:2, or about 1:0.5 to about 1:1.5. In some embodiments, the first ligand and the second ligand may be in a ratio of about 1:1.

In some embodiments, the level of the anti-NS1 antibody and the level of the anti-E-protein antibody may be determined by an enzyme-linked immunosorbent assay (ELISA), a radioimmunoassay (RIA), an immunoprecipitation assay, a radioimmunoprecipitation (RIP) assay, an electrochemiluminescence assay, a chemiluminescence assay, a fluorescence assay, label free—surface plasmon resonance (SPR) (e.g., BIACORE), or gel blotting.

In some embodiments, the first solid support and/or the second solid support may comprise a plurality of beads, a plurality of microparticles, a multiwell plate, a slide, a test tube, a chip, a strip, a sheet, a filter, cross-linked gel supports (e.g., agarose, acrylamide), immobilized resins (e.g., sepharose, dextran, cellulose), microspheres, or any combination thereof.

In some embodiments, the first solid support may comprise a plurality of beads and the second solid support may comprise a plurality of beads. In some embodiments, the plurality of beads of the first solid support and the plurality of beads of the second solid supports may be different, such that the first ligand and the second ligand may be immobilized on the plurality of beads from the first and second solid supports, respectively.

In some embodiments, the first solid support may comprise a plurality of beads and the second solid support may comprise a plurality of beads. In some embodiments, the plurality of beads of the first solid support and the plurality of beads of the second solid supports may be the same, such that the first ligand and the second ligand may be immobilized on the same beads.

In certain embodiments, the first ligand and the second ligand may be covalently coupled to the first solid support and/or the second solid support. In some embodiments, the first ligand and the second ligand may be coupled to the first solid support and/or the second solid support by passive absorption. In some embodiments, the first ligand and second ligand may be coupled directly to the first solid support and/or the second solid support. In some embodiments, the first ligand and the second ligand may be coupled to the first solid support and/or second solid support using an immune-capture, e.g., an anti-polyhistidine antibody may be coupled to the solid support (covalently or by passive adsorption) and binds a histidine “tag” (e.g., 6×HIS) on the ligands.

Methods are also provided for differentiating between biological samples from a subject with Zika virus antibodies and a subject with Non-Zika flavivirus antibodies. Such methods involve (a) detecting the presence or absence of an antibody against (e.g., binding to) a Zika Non-Structural protein 1 (anti-NS1 antibody) and the presence or absence of an antibody against (e.g., binding to) a Zika Envelope protein (anti-E-protein antibody) in a biological sample, wherein the presence of both the anti-NS1 antibody and the anti-E-protein antibody indicates that the sample is a flavivirus-positive biological sample; (b) determining a level of the anti-NS1 antibody and a level of the anti-E-protein antibody in the biological sample; wherein the biological sample is from a subject with Zika-virus antibodies if the level of the anti-NS1 antibody is greater than the level of the anti-E-protein antibody.

Also provided herein is a method for determining if a flavivirus-positive biological sample is from a subject previously infected with a Zika virus or from a subject previously infected with a non-Zika flavivirus, by determining a level of an antibody against a Zika Non-Structural protein 1 (anti-NS1 antibody) and a level of an antibody against a Zika Envelope protein (anti-E-protein antibody) in the flavivirus-positive biological sample; wherein the flavivirus-positive biological sample is from a subject previously infected with Zika-virus if (i) both the anti-NS1 antibody and the anti-E-protein antibody are present in the flavivirus-positive biological sample; and (ii) the level of the anti-NS1 antibody is greater than the level of the anti-E-protein antibody.

In some embodiments, the biological sample may be a body fluid sample selected from the group consisting of whole blood, serum, plasma, urine, saliva, seminal fluid, cerebrospinal fluid, or a combination thereof. Other biological samples that can be used with the present disclosure are provided elsewhere in the current disclosure. In certain embodiments, the biological sample may be plasma.

The methods disclosed herein may also be used to identify suitable donor or donors for use in preparing a hyperimmune composition against other flaviviruses (e.g., Dengue) or against related non-flaviviruses, such as alphaviruses (e.g., Chikungunya virus).

### II. COMPOSITIONS FOR DETECTING MULTIPLE ANTIBODIES

Certain aspects of the application are directed to compositions, e.g., solid supports and solutions, for detecting multiple antibodies in a biological sample (e.g., plasma or serum). These solid supports may be used according to any of the methods disclosed herein. In some embodiments, the composition may comprise a solid support comprising a first ligand and a second ligand, wherein the first ligand binds to a variable region of a first target antibody (e.g., anti-NS1 antibody) and the second ligand binds to a variable region of a second target antibody (e.g., anti-E-protein antibody), wherein the first ligand and the second ligand are immobilized on the solid support.

In some embodiments, the solid support may comprise a glass or plastic structure (e.g., polystyrene or polyvinylidene fluoride) including those treated with protein immobilizing agents such as poly-lysine, nitrocellulose, or porous membranes. Solid supports can also include structures in liquid suspension, such as latex or metal microbeads, including those treated with protein immobilizing agents such as poly-lysine, nitrocellulose, or porous membranes.

The solid supports used in the methods of the present disclosure can be of any kind available in the art, e.g., a bead, a microparticle, a multiwell plate, a slide, a test tube, a chip, a strip, a sheet, a filter, a cross-linked gel support (e.g., agarose, acrylamide), an immobilized resin (e.g., sepharose, dextran, cellulose), a microsphere, or any combination thereof.

In certain aspects, the solid support may be individually identified. Such identification may be possible for example when a plurality of solid supports are separately located in space (e.g., the wells in a microtiter plate, different locations on a chip, different beads, or different locations on a bead) or when they are differently labeled. In some embodiments, a solid support can comprise either discrete small parts of a whole structure (in case of a plate or a chip) or a large number of identical microparticles (e.g., microbeads) that share common characteristics (also referred to as microparticles “subset”).

In some embodiments, the solid supports of the present disclosure may be specifically identified by their specific location, size, diameter, weight, granulometry, labeling, or any combination thereof. Such labeling may include, for example, a fluorochrome, a fluorophore, a chromophore, a radioisotope, a mass tag, or any kind of detectable tag or label which is known in the art.

In some embodiments, the solid support may be made of a material selected from the group consisting of polystyrene, cellulose, nitrocellulose, glass, ceramic, resin, rubber, plastic, silica, silicone, metal, polymer, or any combination thereof. Polymeric materials include brominated polystyrene, polyacrylic acid, polyacrylonitrile, polyamide, polyacrylamide, polyacrolein, polybutadiene, polycaprolactone, polycarbonate, polyester, polyethylene, polyethylene terephthalate, polydimethylsiloxane, polyisoprene, polyurethane, polyvinylacetate, polyvinylchloride, polyvinylpyridine, polyvinylbenzylchloride, polyvinyltoluene, polyvinylidene chloride, polydivinylbenzene, polymethylmethacrylate, polylactide, polyglycolide, poly(lactide-co-glycolide), polyanhydride, polyorthoester, polyphosphazene, polyphosophaze, polysulfone, or any combination thereof, that are acceptable as well. Most of these supports are commercially available. For example, beads from synthetic polymers such as polystyrene, polyacrylamide, polyacrylate, or latex are commercially available from numerous sources such as Bio-Rad Laboratories (Richmond, Calif.) and LKB Produkter (Stockholm, Sweden). Beads formed from natural macromolecules and particles such as agarose, cross-linked agarose, globulin, deoxyribose nucleic acid, and liposomes are commercially available from sources such as Bio-Rad Laboratories, Pharmacia (Piscataway, N.J.), and IBF (France). Beads formed from copolymers of polyacrylamide and agarose are commercially available from sources such as IBF and Pharmacia.

In some embodiments, a solid support may be used in the immunoassays of the present disclosure should be intrinsically identifiable, so that it is possible to determine precisely which antigen is carried by which solid support. Antigen-coupled and identifiable solid supports may then be used as capture reagents for specific human immunoglobulins and contacted with the biological sample of the patient. By analyzing to which solid support the antibodies are bound, it can be determined which antibodies (e.g., anti-NS1 antibody and anti-E-protein antibody) are present in the biological sample.

In some embodiments, a solid support may comprise a plurality of beads, a plurality of microparticles, a multiwell plate, a slide, a test tube, a chip, a strip, a sheet, a filter, a cross-linked gel support (e.g., agarose, acrylamide), an immobilized resin (e.g., sepharose, dextran, cellulose), a microsphere, or any combination thereof. In certain embodiments, the solid support may comprise a plurality of beads wherein the first ligand and the second ligand are immobilized to the same or different beads.

In some embodiments, the first ligand and the second ligand may be immobilized on separate beads that can be individually identified and analyzed. In certain embodiments, the separate beads may be of different sizes, e.g., as described in International Patent Publication No. WO 1999/026067 (Watkins et al.). In other embodiments, the separate beads are labeled with different fluorescent markers, such that they can be individually identified and assessed using e.g., flow cytometry. See, e.g., Vignali, D A, Journal of Immunological Methods 243:243-255 (2000); and Park, M K, et al., Clinical and Diagnostic Laboratory Immunology 7:486-489 (2000). In some embodiments, the separate beads may be labeled (e.g., internally) with different fluorescent markers. In other embodiments, the separate beads may be labeled with different fluorescent markers after the binding of the antibodies to the antigens on the beads.

In some embodiments, the first ligand and the second ligand may be immobilized on the same bead. In certain embodiments, the bead may be color coded into spectrally distinct regions with each region coated with a different antigen. A non-limiting example of such beads are the MICROPLEX microspheres (sold by Luminex), which are carboxylated polystyrene micro-particles that have been color coded into spectrally distinct regions. These regions can be quickly distinguished by an XMAP Instrument allowing for the interrogation of up to 100 different analytes simultaneously from one single sample volume. See U.S. Patent Publication No. 2014/0274762 A1.

In some embodiments, the beads useful for the present disclosure may be magnetic. Magnetic beads are for example commercially available from sources such as Dynal Inc. (Great Neck, N.Y.) or can be prepared using known in the art methods, e.g., as disclosed for example in U.S. Pat. Nos. 4,358,388; 4,654,267; 4,774,265; 5,320,944; and 5,356,713.

In some embodiments, different subsets of beads may be distinguished or separately detected by different labels (e.g., with a fluorochrome, a fluorophore, a chromophore, a radioisotope, a mass tag, or other detectable tag or label).

In some embodiments, the different subsets of beads may be distinguished or separately detected as they are differently fluorescently labeled, as proposed in U.S. Pat. Nos. 5,736,330; 5,981,180; 6,057,107; 6,268,222; 6,449,562; 6,514,295; 6,524,793; and 6,528,165. More precisely, these different subsets may be dyed with different fluorescent dyes, and/or different concentrations of one or more fluorescent dyes. As such, the different subsets may have different fluorescent signatures (e.g., different fluorescent wavelength(s), different fluorescent intensities, etc.) that can be measured and used by a measurement system to determine the subset that individual beads belong to (i.e., to classify the beads according to the subset).

In some embodiments, the first ligand and/or the second ligand may be coupled to the solid supports of the present disclosure by covalent coupling, ionic interactions, electrostatic interactions, or van der Waals forces. In some embodiments, the first ligand and/or the second ligand may be coupled by passive adsorption. In some embodiments, the first ligand and/or the second ligand may be coupled directly to the solid support. In some embodiments, the first ligand and/or the second ligand may be coupled to the solid support using an immune-capture, e.g., an anti-polyhistidine antibody is coupled to the solid support (covalently or by passive adsorption) and binds a histidine “tag” (e.g., 6×HIS) the ligands.

The composition of the present disclosure may also comprise a solution comprising a first ligand and a second ligand, wherein the first ligand binds to the variable region of a first target antibody (e.g., an anti-NS1 antibody) and the second ligand binds to the variable region of a second target antibody (e.g., an anti-E-protein antibody). In some embodiments, the solution may further comprise a plurality of beads that attach to complexes formed by the first ligand bound to the first target antibody (e.g., an anti-NS1 antibody) and the second ligand bound to the second target antibody (e.g., an anti-E-protein antibody).

In certain embodiments, the solution does not comprise a plurality of beads and instead may comprise a first ligand and a second ligand, wherein the first ligand binds to the variable region of the first target antibody (e.g., an anti-NS1 antibody) and the second ligand binds to the variable region of the second target antibody (e.g., an anti-E-protein antibody), wherein the first ligand and the second ligand are conjugated to, e.g., a fluorescent marker. In such embodiments, the fluorescently tagged first ligand and the fluorescently tagged second ligand may be added directly to a biological sample and the formed complexes can be detected with a fluorescence assay (e.g., flow cytometry) without the need for direct binding of the ligands to a solid support.

In some embodiments, the ligand may be a Zika NS1 protein, or a fragment thereof, which binds to the variable region of an anti-NS1 antibody. In some embodiments, the ligand may be a Zika Envelope protein, or a fragment thereof, which binds to the variable region of an anti-E-protein antibody. In some embodiments, the ligand may be a fusion protein comprising at least one epitope that is recognized by a target antibody. In some embodiments, the fusion protein may comprise whole antigens, comprising several epitopes. These epitopes may be linear or conformational epitopes. As used herein, a linear (or sequential) epitope is an epitope that is recognized by antibodies by its linear sequence of amino acids, or primary structure. In contrast, a conformational epitope may be recognized by its specific three-dimensional shape. In some embodiments, the fusion protein of the application may comprise conformational epitopes, as most polyclonal antibodies recognize same.

In some embodiments, the first ligand may comprise a Zika NS1 polypeptide or a fragment thereof. In some embodiments, the second ligand may comprise a Zika E-protein or a fragment thereof. In certain embodiments, the Zika NS1 polypeptide may be a full length protein (e.g., 352 amino acids long) as set forth in SEQ ID NOs: 9-16 or 18. In other embodiments, the Zika NS1 polypeptide may be a fragment or variant thereof, so long as the fragment or variant thereof binds to a variable region of an anti-NS1 antibody. In some embodiments, the Zika NS1 polypeptide may have at least 80%, at least 85%, at least 90%, at least 91%, at least 92%, at least 93%, at least 94%, at least 95%, at least 96%, at least 97%, at least 98%, at least 99%, or 100% amino acid sequence identity to SEQ ID NOs: 9-16 or 18. In some embodiments, the Zika E-protein may be a full-length protein (e.g., 504 amino acids long) as set forth in SEQ ID NOs: 1-8 or 17. In certain embodiments, the Zika E-protein may be a fragment or variant thereof, so long as the fragment or variant thereof binds to a variable region of an anti-E-protein antibody. In some embodiments, the Zika E-protein may have at least 80%, at least 85%, at least 90%, at least 91%, at least 92%, at least 93%, at least 94%, at least 95%, at least 96%, at least 97%, at least 98%, at least 99%, or 100% amino acid sequence identity to SEQ ID NOs: 1-8 or 17.

In some embodiments, the first ligand and/or the second ligand may comprise a protein from a related non-flavivirus, such as alphaviruses (e.g., Chikungunya virus). A composition comprising such a first ligand and a second ligand can be useful in differentiating a flavivirus-positive biological sample from a biological sample that is positive for a related family of viruses.

In certain embodiments, the first ligand and the second ligand may be present at a ratio of about 1:0.1 to about 1:20 or about 0.1:1 to about 20:1. In some embodiments, the first ligand and the second ligand may be present at a ratio of about 1:0.01, about 1:0.05, about 1:0.1, about 1:0.5, about 1:1, about 1:2, about 1:3, about 1:4, about 1:5, about 1:6, about 1:7, about 1:8, about 1:9, about 1:10, about 1:15, or about 1:20. In other embodiments, the first ligand and the second ligand may be present at a ratio of about 1:0.1 to about 1:2, about 1:0.1 to about 1:1.5:, about 1:0.5 to about 1:2, or about 1:0.5 to about 1:1.5. In certain embodiments, the first ligand and second ligand may be present at a ratio of about 1:1.

### III. METHODS FOR DETECTING MULTIPLE ANTIBODIES

The present disclosure also provides methods for detecting multiple antibodies (e.g., anti-NS1 antibody and anti-E-protein antibody) in a biological sample. In some embodiments, the multiple antibodies may be detected using the compositions (e.g., solid supports or solutions) disclosed herein.

Provided herein is a method for detecting two different target antibodies present in a biological sample obtained from one or more subjects. In some embodiments, the method comprises (a) contacting the biological sample with a first solid support bound to a first ligand, which binds to a variable region of a first target antibody; (b) contacting the biological sample with a second solid support bound to a second ligand, which binds to a variable region of a second target antibody; and (c) detecting the presence, absence, amount, level and/or ratio of the two target antibodies by detecting the binding or lack of binding of the first target antibody and the second target antibody to the first ligand and second ligand, respectively. In some embodiments, (a) and (b) may occur simultaneously (e.g., biological sample added to a well comprising both the first solid support and the second solid support). In other embodiments, (a) and (b) may occur at separate times and/or in a separate location. In some embodiments, the first target antibody may be a Zika Non-structural Protein 1 (NS1) antibody (anti-NS1 antibody). In some embodiments, the second target antibody may be a Zika Envelope-protein (E-protein) antibody (anti-E-protein antibody).

The detection methods disclosed herein may also be used to identify biological samples from donors suitable for use in Zika virus hyperimmune compositions and/or to determine if a flavivirus-positive biological sample is from or likely from a subject previously infected with a Zika virus and/or from a subject having detectable levels of antibodies to Zika virus. In another aspect, the detection methods of the present disclosure may be used to differentiate a flavivirus-positive biological sample and a biological sample that is positive for a related (i.e., having large degree of homology with flaviviruses) family of viruses, e.g., alphaviruses (e.g., Chikungunya virus).

To detect the presence of the antibodies that are bound to the solid support and/or beads, any known technology may be used. For example, labeled secondary antibodies recognizing specifically the constant domains of the target antibodies may be used, as described in the Examples. Where the solid support (e.g., beads) are internally labeled with fluorescent markers, it is important to note that the labeling of the detecting-antibodies should be different from the one of the solid support, so as to distinguish between the solid supports that are coupled to antibodies, and those that are not.

In some embodiments, immunoglobulins present in sera from infected animals or humans may be directly conjugated to R-phycoerythrin (R-PE), using a one-step antibody labeling protocol (LIGHTNING-LINK R-Phycoerythrin Conjugation Kit-Innova Biosciences). The hands-on time for the entire procedure is usually 20-30 seconds, and allows the labeling of small quantities of immunoglobulins with up to 100% recovery. This procedure eliminates the need for secondary reagents, such as conjugated anti-species antibodies and streptavidin-R-phycoerythrin, in multiplex-immunoassay experiments.

In some embodiments, the anti-NS1 antibody and/or the anti-E-protein antibody that are bound to the first ligand and the second ligand, respectively, may be detected using an anti-human IgG Fcγ antibody (“secondary antibody”) conjugated to phycoerythrin (PE). In other embodiments, the secondary antibody may be an enzyme-labeled antibody, such that the addition of a substrate for the enzyme produces a detectable signal. In certain embodiments, the enzyme may be selected from the group consisting of alkaline phosphatase, horseradish peroxidase, glucose 6-phosphate dehydrogenase, and β-galactosidase, although other enzymes can be used.

### IV. METHODS OF USE

The passive transfer of antibodies, e.g., hyperimmune globulins, in the form of whole plasma or fractionated preparations, such as gammaglobulins, may be used for both the prophylaxis and treatment of infection in patients, e.g., patients with primary immunodeficiency as well as infections associated with transplantation, chronic leukemia, premature birth, and surgery. See, e.g., Morris, L and Mkhize, N N, PLoS Medicine 14(11):e1002436 (2017); Snydman, D R, et al., New England Journal of Medicine 317:1049, 1987; Kodihalli S, et al., PLoS One 16(9):e106393 (2014); and Kazim, S F, et al., Front Aging Neuroscience 9:71 (2017). In contrast to active immunization, which requires time to produce protective immunity, the passive transfer of antibodies can provide immediate immune protection. See Baxter, D, Occupational Medicine (London) 57(8):552-556 (2007). Such protection may be useful, for instance, in areas of high risk of infection and insufficient time for the body to develop its own immune response, or to reduce the symptoms of ongoing infection.

Not being bound by any one theory, the Zika virus hyperimmune compositions disclosed herein may treat or reduce the risk of a Zika virus infection, related disease or symptoms by increasing the amount of circulating Zika-specific antibodies in the individual. As a result, upon exposure to the Zika virus, the circulating Zika-specific antibodies may neutralize the virus upon exposure.

Accordingly, the present disclosure also relates to the preparation of Zika virus hyperimmune compositions and the use of such compositions to treat a Zika virus infection. In some embodiments, the hyperimmune composition may be derived from one or more individuals who have been positively diagnosed as being Zika probable (i.e., previously having or likely previously having a Zika virus infection), e.g., using the assays and/or methods disclosed herein. In some embodiments, the hyperimmune composition may be derived from one or more individuals who have been positively identified to have elevated levels of anti-Zika antibodies, e.g., using the assays and/or methods disclosed herein. In other embodiments, the hyperimmune composition may be derived from one or more individuals that have been hyper-immunized with one or more Zika virus antigens (e.g., the E-protein, the NS1 protein, and/or whole inactivated or attenuated Zika virus). In some embodiments, at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, at least 5%, at least 6%, at least 7%, at least 8%, at least 9%, or at least 10% of the IgG circulating in the Zika virus exposed and/or hyperimmunized individual or individuals are Zika virus specific.

In one aspect, methods for preparing a Zika virus-specific hyperimmune composition comprises (a) identifying one or more suitable donors according to the methods disclosed herein, and (b) processing a plasma or serum from the one or more suitable donors to provide the Zika virus hyperimmune composition. In some embodiments, the method further comprises purifying a Zika virus-specific antibody (including antigen-binding fragments thereof) from the processed plasma or serum. In some embodiments, the Zika virus-specific antibody may be an IgG antibody. The Zika virus-specific antibody may be purified from the plasma or the serum by any method known in the art (e.g., affinity chromatography or using methods described in Chapter 14, Price et al., Production of Plasma Proteins for Therapeutic Use by Neil Goss). Because the amount of antibody that can be recovered from a single plasma or serum sample can be limited, in some embodiments, the plasma or serum from the suitable donor or donors are pooled into batches prior to the processing. In some embodiments, the plasma or serum, which are pooled into batches, may be from the same donor. In some embodiments, the plasma or serum, which are pooled into batches, may be from multiple donors.

The Zika virus hyperimmune compositions disclosed herein may be used to treat and/or prevent a Zika virus infection or to reduce symptoms associated with a Zika virus infection. In some embodiments, the method of treating, preventing, or reducing the risk of a Zika virus infection in a subject may comprise administering the Zika virus hyperimmune composition as disclosed herein.

Administration of the hyperimmune composition of the present disclosure may increase the circulating antibodies against the Zika virus in a subject, and thereby, confer protective immunity to the subject against the Zika virus. Therefore, in some embodiments, the administration of the hyperimmune composition may treat a subject infected with a Zika virus infection. In other embodiments, the hyperimmune composition may reduce the likelihood of a Zika virus infection, which can be greatly useful for individuals traveling to areas with high Zika virus prevalence. In some embodiments, the administration of the hyperimmune composition may prevent, ameliorate, and/or reduce the symptoms and/or diseases and disorders associated with a Zika virus infection.

In some embodiments, symptoms associated with a Zika virus infection may include: headaches, fever, lethargy, rash, conjunctivitis, myalgia, and arthralgia. In some embodiments, diseases and disorders associated with a Zika virus infection may include neurotropic Guillain-Barre syndrome and congenital microcephaly.

In some embodiments, the hyperimmune composition may be administered to the subject intravenously or intramuscularly. In some embodiments, the hyperimmune composition may be administered with one or more additional therapeutic agent. In some embodiments, the hyperimmune composition of the present disclosure may be administered before or after a Zika virus infection, e.g., within 1 hour, within 2 hours, within 4 hours, within 6 hours, within 12 hours, within 24 hours, within 36 hours, within 48 hours, or within 60 hours before or after infection or after detection of symptoms, or even at a later time.

As described herein, the traditional assays for diagnosing Zika virus infection involves measuring antibody levels of a single Zika antigen. However, because of the highly conserved nature of these antigens, antibodies directed to other flavivirus (e.g., Dengue virus) are cross-reactive to some Zika antigens, resulting in many false positive diagnoses (e.g., a Dengue infected individual having a positive result). Accordingly, the present disclosure provides methods for more accurately diagnosing or identifying a subject or subjects who are presently or previously infected or have previously been exposed to a Zika virus antigen (e.g., previously infected with a Zika virus or exposed to Zika virus antigens (e.g., the E-protein, and/or the NS1 protein, and/or whole inactivated or attenuated Zika virus)) by detecting two types of antibodies. Subjects identified as having elevated antibodies against the Zika virus (e.g., compared to an individual or pool of individuals who have never been exposed to the Zika virus antigen) may be plasma and/or serum donors for preparing a Zika virus hyperimmune composition. In some embodiments, subjects identified as having low or no Zika virus specific antibodies or low or no Zika virus neutralizing antibodies may be treated with a Zika virus hyperimmune composition, e.g., if the subject has an existing, suspected, or possible future Zika virus infection, and/or can be vaccinated with a Zika vaccine.

In some embodiments, the method for identifying a biological sample from a subject or subjects infected with or previously exposed to a Zika virus may comprise contacting the biological sample with a first ligand and a second ligand, wherein the first ligand may bind to a variable region of an anti-NS1 antibody and wherein the second ligand binds to a variable region of an anti-E-protein antibody, wherein if both antibodies are detected and the detected level of anti-NS1 antibody is greater than the detected level of anti-E-protein antibody, the subject is identified as having been infected or previously exposed to Zika virus. In some embodiments, the first ligand may be bound to a first solid support and the second ligand may be bound to a second solid support. In some embodiments, the first solid support may be contacted with a first detectable label and the second solid support may be contacted with a second detectable label, wherein the first detectable label may bind to a constant region of the anti-NS1 antibody and wherein the second detectable label may bind to a constant region of the anti-E-protein antibody.

In some embodiments, the first ligand may comprise a Zika NS1 polypeptide or fragment thereof. In some embodiments, the second ligand may comprise a Zika E-protein polypeptide. In certain embodiments, the Zika NS1 polypeptide may be a full length protein (e.g., 352 amino acids long) as set forth in SEQ ID NOs: 9-16 or 18. In other embodiments, the Zika NS1 polypeptide may be a fragment or variant thereof, so long as the fragment or variant thereof binds to a variable region of an anti-NS1 antibody. In some embodiments, the Zika NS1 polypeptide may have at least 80%, at least 85%, at least 90%, at least 91%, at least 92%, at least 93%, at least 94%, at least 95%, at least 96%, at least 97%, at least 98%, at least 99%, or 100% amino acid sequence identity to SEQ ID NOs: 9-16 or 18. In some embodiments, the Zika E-protein may be a full-length protein (e.g., 504 amino acids long) as set forth in SEQ ID NOs: 1-8 or 17. In certain embodiments, the Zika E-protein may be a fragment or variant thereof, so long as the fragment or variant thereof binds to a variable region of an anti-E-protein antibody. In some embodiments, the Zika E-protein may have at least 80%, at least 85%, at least 90%, at least 91%, at least 92%, at least 93%, at least 94%, at least 95%, at least 96%, at least 97%, at least 98%, at least 99%, or 100% amino acid sequence identity to SEQ ID NOs: 1-8 or 17.

The antibodies bound to the first solid support and/or the second solid support (e.g., plurality of beads) may be detected using any known technology in the art or described elsewhere in the current application.

In some embodiments, the biological sample may be a body fluid sample selected from the group consisting of whole blood, serum, plasma, urine, saliva, seminal fluid, cerebrospinal fluid, or a combination thereof.

As disclosed supra., the methods disclosed herein may also be useful in preparing hyperimmune compositions against other flaviviruses (e.g., Dengue) or against other related non-flaviviruses, e.g., alphaviruses (e.g., Chikungunya virus).

### V. KITS

Provided herein are kits for use in the multianalyte immunoassays described herein, wherein the kits are for e.g., diagnosing (ongoing or previous Zika virus infection) or identifying a suitable plasma donor(s), serum donor(s), pooled serum, or pooled plasma for preparing a Zika virus hyperimmune composition and/or treating, preventing, or reducing a risk for a Zika virus infection. In some embodiments, the kit may comprise one or more containers filled with one or more of the components of the compositions described herein, such as the solid support or the solution of the multianalyte immunoassay described herein. In some embodiments, the kit may comprise the solid support or the solution of the present disclosure and a detectable label. In some embodiment, the detectable label may be detected using any assay known in the art, such as an assay selected from the group consisting of an enzyme-linked immunosorbent assay (ELISA), a radioimmunoassay (RIA), an immunoprecipitation assay, a radioimmunoprecipitation (RIP) assay, an electrochemiluminescence assay, a chemiluminescence assay, or a fluorescence assay. In some embodiments, the kit is for detecting anti-NS1 antibody and anti-E-protein antibody in a body fluid sample and comprises: (a) a solid support(s) or a solution(s) as described herein (b) a first ligand and a second ligand, wherein the first and second ligands bind variable region of an anti-NS1 antibody and an anti-E-protein antibody, respectively; (c) a first detectable label which binds to a constant region of the anti-NS1 antibody; and (d) a second detectable label which binds to a constant region of the anti-E-protein antibody. In some embodiments, (b) may be bound to or present in the solid support or solution, respectively, of (a).

In some embodiment, the kit for detecting anti-NS1 antibody and anti-E-protein antibody in a biological sample comprises: (a) a solid support(s) or a solution(s) as disclosed herein; (b) a first detectable label which binds to a constant region of the anti-NS1 antibody; (c) a second detectable label which binds to a constant region of the anti-E-protein antibody; and (d) a first ligand and a second ligand, wherein the first and second ligands bind variable region of an anti-NS1 antibody and an anti-E-protein antibody, respectively. In some embodiments, the kit may comprise a positive control sample. In some embodiments, (b) is bound to or present in the solid support or solution, respectively, of (a).

### VI. DEFINITIONS

In order that the present disclosure can be more readily understood, certain terms are defined. As used in this application, except as otherwise expressly provided herein, each of the following terms shall have the meaning set forth below. Additional definitions are set forth throughout the application.

As used herein, “a” or “an” means one or more unless otherwise specified.

As used herein, “and/or” is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, the term “about” is understood as within a range of normal tolerance in the art and not more than ±10% of a stated value. By way of example only, about 50 means from 45 to 55 including all values in between. As used herein, the phrase “about” a specific value also includes the specific value, for example, about 50 includes 50.

The terms “antibody,” “antibodies,” “immunoglobulin,” “immune globulin,” “immune globulins,” and “immunoglobulins” can be used interchangeably herein and refer to a molecule with an antigen binding site that specifically binds an antigen. The terms as used herein include whole antibodies and any antigen binding fragments (i.e., “antigen-binding fragments”) or single chains thereof. An “antibody” refers, in one embodiment, to a glycoprotein comprising at least two heavy (H) chains and two light (L) chains inter-connected by disulfide bonds, or an antigen-binding fragment thereof. In another embodiment, an “antibody” refers to a single chain antibody comprising a single variable domain, e.g., VHH domain. Each heavy chain is comprised of a heavy chain variable region (abbreviated herein as VH) and a heavy chain constant region. In certain naturally-occurring antibodies, the heavy chain constant region is comprised of three domains, CH1, CH2, and CH3. In certain naturally-occurring antibodies, each light chain is comprised of a light chain variable region (abbreviated herein as VL) and a light chain constant region. The light chain constant region is comprised of one domain, CL.

As used herein, the term “target antibody” refers to an antibody that is being detected. In some embodiments, the target antibody is an anti-NS1 antibody and/or an anti-E-protein antibody.

As used herein, the terms “antigen” and “immunogen” refer to any substance that is capable of inducing an adaptive immune response. An antigen may be whole cell (e.g., bacterial cell), virus, fungus, or an antigenic portion or component thereof. Non-limiting examples of antigens for the present disclosure include a Zika virus Envelope protein or a fragment or a variant thereof, or a Zika virus Non-structural 1 protein or a fragment or a variant thereof.

As used herein, the term “epitope” designates a particular molecular surface feature of an antigen, for example a fragment of an antigen, which is capable of being bound by at least one antibody. Antigens usually present several surface features that can act as points of interaction for specific antibodies. Any such distinct molecular feature constitutes an epitope. On a molecular level, an epitope therefore corresponds to a particular molecular surface feature of an antigen (for example a fragment of an antigen) which is recognized and bound by a specific antibody.

As used herein, the phrase “viral infection” describes a diseased state in which a virus (e.g., a Zika virus) invades a cell and uses the cell's machinery to multiply or replicate, ultimately resulting in the release of new viral particles. This release results in the infection of other cells by the newly produced particles. Latent infection by certain viruses is also a possible result of viral infection.

As used herein, the term “flavivirus” refers to viruses belonging to the genus Flavivirus of the family Flaviviridae. According to virus taxonomy, about 50 viruses including, e.g., Zika, Hepatitis C (HCV), Yellow Fever, Dengue, Japanese Encephalitis, West Nile, and related flaviviruses are members of this genus. The viruses belonging to the genus Flavivirus are referred to herein as flaviviruses. Currently, these viruses are predominantly in East, Southeast and South Asia and Africa, although they may be found in other parts of the world.

As used herein, the term “alphavirus” refers to any of the RNA viruses included within the genus Alphavirus. Descriptions of the members of this genus are contained in Strauss and Strauss, Microbiological Reviews, 58:491-562 (1994). Examples of alphaviruses include Aura virus, Bebaru virus, Cabassou virus, Chikungunya virus, Eastern equine encephalomyelitis virus, Fort morgan virus, Getah virus, Kyzylagach virus, Mayoaro virus, Middleburg virus, Mucambo virus, Ndumu virus, Pixuna virus, Tonate virus, Triniti virus, Una virus, Western equine encephalomyelitis virus, Whataroa virus, Sindbis virus (SIN), Semliki forest virus (SFV), Venezuelan equine encephalomyelitis virus (VEE), and Ross River virus.

As used herein, the term “Zika virus” comprises any Zika virus, irrespective of strain or origin. In some embodiments, the term relates to a Zika virus from an African or an Asian lineage. In other embodiments, the term ‘Zika virus’ comprises a Zika virus strain selected from the group consisting of (i) strain PLCal_ZV-Thailand (GenBank Accession No. KF993678); (ii) strain PRVABC59-Puerto Rico (GenBank Accession No. KU501215); (iii) strain IbH_30656-Nigeria (GenBank Accession No. HQ234500); (iv) strain MR 766-Uganda (GenBank Accession No. LC002520); (v) strain FSS13025-Cambodia (GenBank Accession No. JN860885); (vi) strain SPH2015-Brazil (GeneBank Accession No. KU321639); (vii) strain Brazil_ZKV2015 (GenBank Accession No. KU497555); and (viii) strain H/PF/2013-Polynesia (GenBank Accession No. KJ776791).

In the context of the present disclosure, the term “Zika virus protein” or “Zika virus polypeptide” comprises an individual structural (i.e., capsid (C), precursor membrane (prM), and envelope (E)) or non-structural Zika virus protein (i.e., NS1, NS2A, NS2B, NS3, NS4A, NS4B, and NS5). The Zika virus protein can be a full-length protein or a fragment or a variant thereof.

Amino acid sequences of exemplary Zika virus E proteins are set forth as SEQ ID NO: 1 (Brazil_ZKV2015); SEQ ID NO: 2 (SPH2015-Brazil); SEQ ID NO: 3 (FSS13025-Cambodia); SEQ ID NO: 4 (IbH_30656-Nigeria); SEQ ID NO: 5 (H/PF/2013-Polynesia); SEQ ID NO: 6 (PRVABC59-Puerto Rico); SEQ ID NO: 7 (PLCal_ZV-Thailand); and SEQ ID NO: 8 (MR 766-Uganda).

Amino acid sequences of exemplary Zika virus NS1 proteins are set forth as SEQ ID NO: 9 (Brazil_ZKV2015); SEQ ID NO: 10 (SPH2015-Brazil); SEQ ID NO: 11 (FSS13025-Cambodia); SEQ ID NO: 12 (IbH_30656-Nigeria; SEQ ID NO: 13 (H/PF/2013-Polynesia); SEQ ID NO: 14 (PRVABC59-Puerto Rico); SEQ ID NO: 15 (PLCal_ZV-Thailand); and SEQ ID NO: 16 (MR 766-Uganda).

The term “diagnosis” or “diagnosing” as used herein refers to methods that can be used to confirm or determine the likelihood of whether a patient is suffering from or had previously suffered from a given disease or condition, e.g., a Zika virus infection. The term “diagnosis” or “diagnosing” does not refer to the ability to determine the presence or absence of exposure to a particular disease or disorder with 100% accuracy, or even that a given course or outcome is more likely to occur than not. Instead, the skilled artisan will understand that the term “diagnosis” refers to an increased probability that a subject has or previously had a certain disease or disorder (e.g., a Zika virus infection).

In some embodiments, the term “identify” or “identifying” a subject(s) or a donor(s) is used to refer to diagnosing a subject(s) or a donor(s) as having previously been infected (or currently infected) with a Zika virus or exposed to a Zika virus antigen (e.g., NS1 protein or E protein).

As used herein, a “detectable label” is a molecule or a combination of molecules that can be used to specifically recognize a target. In some embodiments, the target comprises a complex formed by the binding of an immunologic determinant (e.g., an antigen) to an antigen binding molecule (e.g., an antibody). In some embodiments, the detectable label is conjugated directly or indirectly to a “marker,” which provides a detectable signal for a period of time, e.g., at least the time period during which a signal is to be observed or measured. The marker can be detectable by itself (e.g. radioisotope labels or fluorescent labels) or can catalyze chemical alteration of a substrate compound or composition which is detectable, e.g., in the case of an enzymatic label. In some embodiments, the detectable label comprises a radioisotope, fluorophore, chromophore, enzyme, dye, metal ion, or ligand (e.g., biotin, avidin, streptavidin, haptens, or quantum dots).

As used herein, a “solid support” refers to a structure for immobilization of a molecule or combination of molecules. In some embodiments, the solid support is used to immobilize a ligand (e.g., a Zika antigen).

As used herein, the terms “microparticles,” “microspheres,” or “microbeads” are used interchangeably and bear equivalent meanings as they refer to small particles with overall diameter that falls essentially in the micrometer range.

The terms “nanospheres,” “nanoparticles,” or “nanobeads” refer to smaller particles with overall size that falls essentially in the nanometer range. As used herein the general term particles, spheres, or beads refers both to microparticles and nanoparticles, which can effectively serve as solid supports in the methods of the application.

As used herein, a “subset of,” e.g., beads or microparticles, corresponds to a plurality within a population, e.g., of beads or microparticles, having the same characteristics. In certain embodiments, each subset of beads or microparticles can be distinguishable from other subsets of the population of beads or microparticles, respectively, by at least one characteristic (e.g., location, size, diameter, weight, granulometry, and/or labeling).

As used herein, the term “immunoassay” refers to an assay that detects, determines, identifies, characterizes, quantifies, or otherwise measures the presence or concentration of a macromolecule or a small molecule through the use of an antibody or an antigen. The molecule detected by the immunoassay can be referred to as an “analyte.” Analytes in biological samples (e.g., serum or plasma) can be measured using immunoassays disclosed herein. In some embodiments, the immunoassay includes, for example, direct or competitive binding assays using techniques such as western blots, radioimmunoassays, ELISA (enzyme linked immunosorbent assay), “sandwich” immunoassays, immunoprecipitation assays, fluorescent immunoassays, and protein A immunoassays.

As used herein, the term “ligand” refers to a molecule capable of binding or otherwise recognizing a target (e.g., an antibody). Examples of such ligands include, but are not limited to, a small peptide, a polypeptide, a protein and the like, that specifically bind the desired target antibodies.

As used herein, the term “immobilized” means coupled to a support. In some embodiments, immobilized refers to a ligand (e.g., the Zika virus NS1 and/or Envelope protein) which is coupled to a solid support so that the ligand does not migrate. In some embodiments, immobilized ligands are coupled by covalent coupling, ionic interactions, electrostatic interactions, or van der Waals forces. In other embodiments, the immobilized ligands are coupled by passive adsorption. In some embodiments, the ligands are coupled directly to the solid support. In some embodiments, the ligands are coupled to the solid support using an immune-capture, e.g., an anti-polyhistidine antibody is coupled to the solid support (covalently or by passive adsorption) and binds the ligands.

In some embodiments, the ligands disclosed herein (e.g., Zika virus NS1 and/or Envelope protein) are immobilized directly to a solid support. In other embodiments, the ligands are immobilized indirectly, for example, by immobilizing a non-target antibody or other intermediate entity having affinity to the ligands, followed by formation of a complex to the effect that the ligand-antibody complex is immobilized. Various ways to immobilize molecules are described in the literature, for example in Kim, D and Herr, A E, Biomicrofluidics 7(4):41501 (2013). In addition, various reagents and kits for immobilization reactions are commercially available, for example, from Pierce Biotechnology.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to include the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide can be derived from a natural biological source or produced by recombinant technology, but is not necessarily translated from a designated nucleic acid sequence. It may be generated in any manner, including by chemical synthesis.

Also included as polypeptides and immunogens of the present application are fragments, derivatives, analogs, or variants of the foregoing polypeptides, and any combination thereof. The terms “fragment,” “variant,” “derivative,” and “analog” polypeptides of the present application include any polypeptides that retain at least some of the properties of the corresponding polypeptide of the application. Fragments of polypeptides of the present disclosure include proteolytic fragments, as well as deletion fragments, in addition to specific antibody binding fragments discussed elsewhere herein. Variant polypeptides of the present application include fragments and also polypeptides with altered amino acid sequences due to amino acid substitutions, deletions, or insertions. Variants may occur naturally or be non-naturally occurring. Non-naturally occurring variants may be produced using art-known mutagenesis techniques. Variant polypeptides may comprise conservative or non-conservative amino acid substitutions, deletions, or additions. Variant polypeptides can also be referred to herein as “polypeptide analogs.” As used herein, a “derivative” of a polypeptide refers to a subject polypeptide having one or more residues chemically derivatized by reaction of a functional side group. Also included as “derivatives” are those peptides that contain one or more naturally occurring amino acid derivatives of the twenty standard amino acids. Derivatives of polypeptides of the present application can include polypeptides that have been altered so as to exhibit additional features not found on the reference polypeptide of the application. As used herein, the percent “sequence identity” between two sequences is a function of the number of identical positions shared by the sequences (i.e., % homology=# of identical positions/total # of positions×100), taking into account the number of gaps, and the length of each gap, which may be introduced for optimal alignment of the two sequences.

A “binding molecule” or “antigen binding molecule” of the present application refers in its broadest sense to a molecule that specifically binds an antigenic determinant of an antigen.

As used herein, the term “biological sample” refers to any samples which have been obtained from a subject (e.g., a mammalian subject, e.g., a human, bovine, ovine, or equine). In some embodiments, the biological sample contains antibodies. In some embodiments, the biological sample is a biological fluid, for example an unfiltered biological fluid such as urine, cerebrospinal fluid, pleural fluid, synovial fluid, peritoneal fluid, amniotic fluid, gastric fluid, blood, serum, plasma, lymph fluid, interstitial fluid, saliva, physiological secretions, tears, mucus, sweat, milk, semen, seminal fluid, vaginal secretions, fluid from ulcers and other surface eruptions, blisters, and abscesses. In some embodiments, a biological sample also refers to an extract of tissues including biopsies of normal, malignant, and suspect tissues or any other constituents of the body which may contain antibodies. The said biological sample can be pre-treated prior to use, such as preparing plasma from blood, diluting viscous fluids, or the like; methods of treatment can involve purification, filtration, distillation, concentration, inactivation of interfering compounds, and the addition of reagents. In some embodiments, the biological sample is whole blood or a blood product such as plasma or serum.

As used herein, “blood product” refers to a therapeutic substance prepared from blood. In some embodiments, the blood product is plasma, serum, immunoglobulins, or any combination thereof.

As used herein, the terms “treat,” “treating,” and “treatment” refer to administering a therapy in an amount, manner, or mode effective to improve a condition, symptom, or parameter associated with a disease or disorder (e.g., Zika virus infection). Thus, “treating” a Zika virus infection means inhibiting or preventing the replication of the virus, inhibiting, or preventing viral transmission, and/or ameliorating, alleviating, or otherwise improving the symptoms of a disease or condition caused by or associated with the virus. In some embodiments, the treatment can be considered therapeutic if there is a reduction in viral load, and/or a decrease in mortality and/or morbidity.

As used herein, the term “reducing the risk of a Zika virus infection” refers to decreasing the likelihood or probability of developing a disease, disorder, or symptom associated with a Zika virus infection in a subject, wherein the subject is, for example a subject who is at risk for developing such a disease, disorder, or symptom.

The term “therapeutically effective amount” as used herein refers to an amount of a therapeutic agent or a composition comprising a therapeutic agent (e.g., a hyperimmune composition), alone or in combination with another therapeutic agent, effective to treat or reduce symptoms, or reduce the risk, potential, possibility or occurrence of a disease or disorder (e.g., a Zika virus infection) in a subject. A therapeutically effective amount can include an amount of a therapeutic agent or a composition comprising a therapeutic agent (e.g., a hyperimmune composition), alone or in combination with another therapeutic agent, that provides some improvement or benefit to a subject having or at risk of having a Zika virus infection.

As used herein, “administering” refers to the physical introduction of a therapeutic agent or a composition comprising a therapeutic agent (e.g., a hyperimmune composition) to a subject, using any of the various methods and delivery systems known to those skilled in the art. The different routes of administration for antibodies described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, intratracheal, pulmonary, subcutaneous, subcuticular, intraarticular, subcapsular, subarachnoid, intraventricle, intravitreal, epidural, and intrasternal injection and infusion, as well as in vivo electroporation. Alternatively, an antibody described herein can be administered via a non-parenteral route, such as a topical, epidermal or mucosal route of administration, for example, intranasally, orally, vaginally, rectally, sublingually, or topically. Administering can also be performed, for example, once, a plurality of times, and/or over one or more extended periods.

As used herein, the term “vaccine” refers to a prophylactic or therapeutic material providing at least one antigen, preferably an immunogen. The antigen or immunogen may be derived from any material that is suitable for vaccination. For example, the antigen or immunogen may be derived from a pathogen, such as from bacteria or virus particles etc., or from a tumor or cancerous tissue. The antigen or immunogen stimulates the body's adaptive immune system to provide an adaptive immune response.

As used herein, the term “Immunized” means sufficiently vaccinated to achieve a protective immune response.

As used herein, the term “hyperimmune” refers to a state of having an elevated level of antibodies to a target, e.g., against a Zika virus, compared to a reference level (e.g., level of anti-Zika virus antibodies in normal source donor comprising non-specific antibodies). In some embodiments, the elevated level of antibodies to a target is generated from exposure to the target virus. In another embodiment, the elevated level of antibodies is generated from donor stimulation (e.g., administration of a vaccine to the target). In some embodiments, the antibodies disclosed herein are immune globulins.

As used herein, the term “hyperimmune composition” (e.g., Zika virus hyperimmune composition) refers to a composition comprising antibodies to specific antigens, e.g., polyclonal antibodies, obtained from plasma and/or serum. In some embodiments, the hyperimmune composition is enriched with antibodies specific to one or more particular epitopes of a Zika virus (e.g., anti-NS1 and/or anti-E-protein antibodies). In some embodiments, the hyperimmune composition disclosed herein is prepared from a plasma and/or serum obtained from an individual or pool of individuals with elevated levels of anti-Zika virus antibodies. In some embodiments, the individual or pool of individuals disclosed herein have elevated levels of anti-Zika virus antibodies due to previous exposure to a Zika virus antigen (e.g., an individual or pool of individuals previously infected with a Zika virus). In some embodiments, the individual or pool of individuals disclosed herein have elevated levels of anti-Zika virus antibodies due to intentional stimulation of the immune response (e.g., administration of a Zika vaccine). In some embodiments, the antibodies disclosed herein are immune globulins.

As used herein, the term “hyperimmunization” or “hyperimmunized” refer to a state of immunity that is greater than normal (e.g., non-infected subjects, e.g., healthy subjects) and results in a higher titer than normal number of antibodies to an antigen. In some embodiments, hyperimmunization can be the result of a previous infection with the Zika virus, such that the individual or pool of individuals have higher titer of antibodies against the Zika virus Envelope protein and/or the NS1 protein compared to an individual or pool of individuals who have never been infected with a Zika virus. In some embodiments, hyperimmunization can involve the repeated administration of a single antigen (e.g., Zika virus Envelope protein or the NS1 protein) or multiple antigens of a given virus (e.g., both Zika virus Envelope and the NS1 proteins) to one or more subjects to generate an enhanced immune response (e.g., higher titer of antibodies against Zika virus Envelope protein and/or NS1 protein compared to a subject not exposed to the antigen).

As used herein, the term “passive immunization” refers to conferral of immunity by the administration, by any route, of exogenously produced immune molecules (e.g., antibodies) into a subject. Passive immunization differs from “active” immunization, where immunity is obtained by introduction of an immunogen into an individual to elicit an immune response.

As used herein, the terms “pooled plasma,” “pooled plasma samples,” and “pooled plasma composition” refer to a mixture of two or more plasma samples and/or a composition prepared from the same (e.g., immunoglobulin). In some embodiments, the plasma samples are obtained from a single donor. In some embodiments, the plasma samples are obtained from multiple donors. Elevated titer of a particular antibody or set of antibodies in pooled plasma reflects the elevated titers of the antibody samples that make up the pooled plasma. For example, plasma samples can be obtained from donors or subjects that have been vaccinated (e.g., with a vaccine) or donors or subjects that have high titers of antibodies to a Zika virus antigen (e.g., after a Zika virus infection) as compared to the antibody level(s) found in a population of subjects never infected with Zika virus or the population as a whole. Upon pooling of the plasma samples, a pooled plasma composition is produced (e.g., that has an elevated titer of antibodies specific to a particular antigen). Pooled plasma compositions can be used to prepare immunoglobulin (e.g., that is subsequently administered to a subject) via methods known in the art (e.g., fractionation, purification, isolation, etc.). The present disclosure provides that pooled plasma compositions, pooled serum compositions, and immunoglobulin prepared from same can be administered to a subject to provide prophylactic and/or therapeutic benefits to the subject. Accordingly, the term pooled plasma composition or pooled serum composition can refer to immunoglobulin prepared from pooled plasma or pooled serum samples, respectively.

As used herein, the terms “subject” or “individual,” which terms are used interchangeably herein, refers to any subject, particularly a mammalian subject, particularly humans. Other subjects can include non-human primates, cattle, dogs, cats, guinea pigs, rabbits, rats, mice, horses, goats, sheep, and so on. In certain embodiments, the subject can be a pregnant mammal, and in particular embodiments, a pregnant human female. In some embodiments, the subject is a patient, for whom prophylaxis or therapy is desired. In some embodiments, the subject is a donor. In some embodiments, the terms “subject” or “individual” can refer to a single subject or individual. In other embodiments, the terms “subject” or “individual” can refer to multiple subjects or individuals.

As used herein, the term “donor” refers to a subject who is a source of a biological material, e.g., blood or blood product. In some embodiments, the donor is a mammal, e.g., a human, a non-human primate, or a horse. In some embodiments, the donor is a plasma and/or serum donor. In some embodiments, the term “donor” can refer to a single donor. In other embodiments, the term “donor” can refer to multiple donors.

As used herein, the terms “at risk for infection” and “at risk for disease” refer to a subject that is predisposed to experiencing a particular infection or disease (e.g., Zika virus infection). This predisposition may be genetic (e.g., a particular genetic tendency to experience the disease, such as heritable disorders), or due to other factors (e.g., immunosuppression, compromised immune system, immunodeficiency, environmental conditions, exposures to detrimental compounds present in the environment, etc.). Thus, it is not intended that the present disclosure be limited to any particular risk (e.g., a subject may be “at risk for disease” simply by being exposed to and interacting with other people).

### VII. EMBODIMENTS

For further illustration, additional non-limiting embodiments of the present disclosure are set forth below.

For example, embodiment A1, is an in vitro method for identifying a donor for use in preparing a Zika virus hyperimmune composition, comprising:

determining a level of an antibody against a Zika Non-Structural protein 1 (anti-NS1 antibody) and a level of an antibody against a Zika Envelope protein (anti-E-protein antibody) in a biological sample from a potential donor;

wherein the potential donor is the donor for use in preparing a Zika virus hyperimmune composition if (i) both the anti-NS1 antibody and the anti-E-protein antibody are present in the biological sample; and (ii) the ratio of the level of the anti-NS1 antibody to the level of the anti-E-protein antibody is greater than about 0.6.

Embodiment A2 is the method of embodiment A1, wherein the level of the anti-NS1 antibody in the biological sample is at least 20% of a level of the anti-NS1 antibody in a positive control sample obtained from one or more individuals previously infected with Zika virus.

Embodiment A3 is the method of embodiment A1 or A2, wherein the biological sample is contacted with a first ligand and a second ligand, wherein the first ligand binds to a variable region of the anti-NS1 antibody and wherein the second ligand binds to a variable region of the anti-E-protein antibody.

Embodiment A4 is the method of embodiment A3, wherein the first ligand is bound to a first solid support and wherein the second ligand is bound to a second solid support.

Embodiment A5 is the method of embodiment A4, wherein the first solid support is contacted with a first detectable label and the second solid support is contacted with a second detectable label, wherein the first detectable label binds to a constant region of the anti-NS1 antibody and wherein the second detectable label binds to a constant region of the anti-E-protein antibody.

Embodiment A6 is the method of any one of embodiments A3 to A5, wherein the first ligand comprises a Zika NS1 polypeptide or antibody binding fragment thereof.

Embodiment A7 is the method of embodiment A6, wherein the Zika NS1 polypeptide or antibody binding fragment thereof comprises a sequence having at least 80%, at least 85%, at least 90%, at least 91%, at least 92%, at least 93%, at least 94%, at least 95%, at least 96%, at least 97%, at least 98%, at least 99%, or 100% amino acid sequence identity to a sequence selected from SEQ ID NOs: 9-16 and 18.

Embodiment A8 is the method of any one of embodiments A3 to A7, wherein the second ligand comprises a Zika E-protein polypeptide or antibody binding fragment thereof.

Embodiment A9 is the method of embodiment A8, wherein the Zika E-protein polypeptide or antibody binding fragment thereof comprises a sequence having at least 80%, at least 85%, at least 90%, at least 91%, at least 92%, at least 93%, at least 94%, at least 95%, at least 96%, at least 97%, at least 98%, at least 99%, or 100% amino acid sequence identity to a sequence selected from SEQ ID NOs: 1-8 and 17.

Embodiment A10 is the method of any one of embodiments A1 to A9, wherein the donor for use in preparing a Zika virus hyperimmune composition was not previously infected with a non-Zika flavivirus.

Embodiment A11 is the method of any one of the preceding embodiments further comprising collecting plasma and/or serum from the donor for preparing the Zika virus hyperimmune composition.

Embodiment A12 is the method of embodiment A11 further comprising preparing immunoglobulin from the plasma and/or serum collected from the donor.

Embodiment A13 is the method of embodiment A11 or A12 further comprising pooling the collected plasma, collected serum, or prepared immunoglobulin for preparing the Zika virus hyperimmune composition.

Embodiment A14 is the method of any one of embodiments A11 to A13 further comprising processing the pooled plasma, serum, or immunoglobulin for preparing the Zika virus hyperimmune composition.

Embodiment B1 is a method of preparing a Zika virus hyperimmune composition, the method comprising:

(a) identifying the donor for use in preparing the Zika virus hyperimmune composition according to any one of embodiments 1 to 10;

(b) collecting plasma and/or serum from the donor;

(c) pooling the collected plasma and/or serum; and

(d) processing the pooled plasma and/or serum.

Embodiment B2 is the method of embodiment B1, wherein the Zika virus hyperimmune composition comprises the processed plasma and/or serum of (d).

Embodiment C1 is a method for differentiating between Zika virus antibodies and Non-Zika flavivirus antibodies in a biological sample from a subject comprising:

(a) detecting the presence or absence of antibodies against a Zika Non-Structural protein 1 (anti-NS1 antibody) and the presence or absence of antibodies against a Zika Envelope protein (anti-E-protein antibody) in a biological sample, wherein the presence of both the anti-NS1 antibodies and the anti-E-protein antibodies indicates that the sample is a flavivirus-positive biological sample;

(b) determining a level of the anti-NS1 antibody and a level of the anti-E-protein antibody in the flavivirus positive biological sample;

wherein the flavivirus positive biological sample is from a subject with Zika-virus antibodies if the level of the anti-NS1 antibody is greater than the level of the anti-E-protein antibody.

Embodiment C2 is the method of embodiment C1, wherein the ratio of the level of the anti-NS1 antibody to the level of the anti-E-protein antibody is greater than about 0.6.

Embodiment C3 is the method of embodiment C1, wherein the flavivirus-positive biological sample is from a subject with Non-Zika flavivirus antibodies if the level of the anti-E-protein antibody is greater than the level of the anti-NS1-protein antibody.

Embodiment C4 is the method of any one of embodiments C1 to C3, wherein the biological sample is contacted with a first ligand and a second ligand, wherein the first ligand binds to a variable region of the anti-NS1 antibodies and wherein the second ligand binds to a variable region of the anti-E-protein antibodies.

Embodiment C5 is the method of embodiment C4, wherein the first ligand is bound to a first solid support and wherein the second ligand is bound to a second solid support.

Embodiment C6 is the method of embodiment C5, wherein the first solid support is contacted with a first detectable label and the second solid support is contacted with a second detectable label, wherein the first detectable label binds to a constant region of the anti-NS1 antibody and wherein the second detectable label binds to a constant region of the anti-E-protein antibody.

Embodiment C7 is the method of any one of embodiments C4 to C6, wherein the first ligand comprises a Zika NS1 polypeptide or antibody binding fragment thereof.

Embodiment C8 is the method of embodiment C7, wherein the Zika NS1 polypeptide or antibody binding fragment thereof comprises a sequence having at least 80%, at least 85%, at least 90%, at least 91%, at least 92%, at least 93%, at least 94%, at least 95%, at least 96%, at least 97%, at least 98%, at least 99%, or 100% amino acid sequence identity to a sequence selected from SEQ ID NOs: 9-16 and 18.

Embodiment C9 is the method of any one of embodiments C4 to C8, wherein the second ligand comprises a Zika E-protein polypeptide or antibody binding fragment thereof.

Embodiment C10 is the method of embodiment C9, wherein the Zika E-protein polypeptide or antibody binding fragment thereof comprises a sequence having at least 80%, at least 85%, at least 90%, at least 91%, at least 92%, at least 93%, at least 94%, at least 95%, at least 96%, at least 97%, at least 98%, at least 99%, or 100% amino acid sequence identity to a sequence selected from SEQ ID NOs: 1-8 and 17.

Embodiment C11 is the method of any one of embodiments B1 to C10, wherein the Non-Zika flavivirus is dengue virus, Hepatitis C (HCV) virus, Yellow Fever virus, Japanese Encephalitis virus, or West Nile virus.

Embodiment C12 is the method of any one of the preceding embodiments, wherein the level of the anti-NS1 antibody and the level of the anti-E-protein antibody are determined by an enzyme-linked immunosorbent assay (ELISA), a radioimmunoassay (RIA), an immunoprecipitation assay, a radioimmunoprecipitation (RIP) assay, an electrochemiluminescence assay, a chemiluminescence assay, a fluorescence assay, label free—surface plasmon resonance (SPR), or gel blotting.

Embodiment D1 is an in vitro method for detecting two target antibodies present in a biological sample comprising:

(a) contacting the biological sample with a first solid support bound to a first ligand, which binds to a variable region of a first target antibody, wherein the first target antibody is a Zika Non-structural Protein 1 (NS1) antibody (anti-NS1 antibody);

(b) contacting the biological sample with a second solid support bound to a second ligand, which binds to a variable region of a second target antibody, wherein the second target antibody is a Zika Envelope-protein (E-protein) antibody (anti-E-protein antibody); and

(c) detecting the presence or absence of the two target antibodies by detecting the binding or lack of binding of the first target antibody and the second target antibody to the first ligand and second ligand, respectively.

Embodiment D2 is the method of embodiment D1, wherein the first ligand comprises a Zika NS1 polypeptide or antibody binding fragment thereof.

Embodiment D3 is the method of embodiment D2, wherein the Zika NS1 polypeptide or antibody binding fragment thereof comprises a sequence having at least 80%, at least 85%, at least 90%, at least 91%, at least 92%, at least 93%, at least 94%, at least 95%, at least 96%, at least 97%, at least 98%, at least 99%, or 100% amino acid sequence identity to a sequence selected from SEQ ID NOs: 9-16 and 18.

Embodiment D4 is the method of any one of embodiments D1 to D3, wherein the second ligand comprises a Zika E-protein polypeptide or antibody binding fragment thereof.

Embodiment D5 is the method of embodiment D4, wherein the Zika E-protein polypeptide or antibody binding fragment thereof comprises a sequence having at least 80%, at least 85%, at least 90%, at least 91%, at least 92%, at least 93%, at least 94%, at least 95%, at least 96%, at least 97%, at least 98%, at least 99%, or 100% amino acid sequence identity to a sequence selected from SEQ ID NOs: 1-8 and 17.

Embodiment D6 is the method of any one of embodiments D1 to D5, further comprising contacting the first solid support with a first detectable label and the second solid support with a second detectable label, wherein the first detectable label binds to a constant region of the first target antibody and wherein the second detectable label binds to a constant region of the second target antibody.

Embodiment D7 is the method of any one of embodiments D1 to D6, wherein the anti-NS1 antibody and the anti-E-protein antibody are detected by an enzyme-linked immunosorbent assay (ELISA), a radioimmunoassay (RIA), an immunoprecipitation assay, a radioimmunoprecipitation (RIP) assay, an electrochemiluminescence assay, a chemiluminescence assay, or a fluorescence assay.

Embodiment D8 is the method of any one of embodiment A4 to B2 and C5 to D7, wherein the first and/or the second solid support comprises a plurality of beads, a plurality of microparticles, a multiwell plate, a slide, a test tube, a chip, a strip, a sheet, a filter, cross-linked gel supports, immobilized resins, microspheres, or a combination thereof.

Embodiment D9 is the method of embodiment D8, wherein the first solid support comprises a plurality of beads and the second solid support comprises a plurality of beads, wherein the plurality of beads of the first and second solid supports are different, such that the first ligand and the second ligand are immobilized on separate beads.

Embodiment D10 is the method of embodiment D9, wherein the first solid support comprises a plurality of beads and the second solid support comprises a plurality of beads, wherein the plurality of beads of the first and second solid supports are the same, such that the first ligand and the second ligand are immobilized on the same beads.

Embodiment D11 is the method of any one of embodiments A4 to B2 and C5 to D10, wherein the first ligand and the second ligand are covalently coupled to the first and/or second solid support.

Embodiment D12 is the method of any one of embodiments A4 to B2 and C5 to D10, wherein the first ligand and the second ligand are coupled to the first and/or second solid support by passive absorption.

Embodiment D13 is the method of any one of the preceding embodiments, wherein the biological sample is a body fluid sample selected from the group consisting of whole blood, serum, plasma, urine, saliva, seminal fluid, cerebrospinal fluid, and a combination thereof.

Embodiment D14 is the method of embodiment D13, wherein the biological sample is serum or plasma.

Embodiment E1 is a solid support comprising a first ligand and a second ligand, wherein the first ligand binds to a variable region of a Zika Non-structural Protein 1 (NS1) antibody (anti-NS1 antibody) and the second ligand binds to a variable region of a Zika Envelope-protein antibody (E-protein) (anti-E-protein antibody), wherein the first ligand and the second ligand are immobilized on the solid support.

Embodiment E2 is the solid support of embodiment E1, which comprises a plurality of beads, a plurality of microparticles, a multiwell plate, a slide, a test tube, a chip, a strip, a sheet, a filter, cross-linked gel supports, immobilized resins, microspheres, or a combination thereof.

Embodiment E3 is the solid support of embodiment E1, which comprises a plurality of beads, wherein the first ligand and the second ligand are immobilized on separate beads.

Embodiment E4 is the solid support of embodiment E1, which comprises a plurality of beads, wherein the first ligand and the second ligand are immobilized on the same beads.

Embodiment E5 is the solid support of any one of embodiments E1 to E4, wherein the first ligand and the second ligand are covalently coupled to the solid support.

Embodiment E6 is the solid support of any one of embodiments E1 to E4, wherein the first ligand and the second ligand are coupled to the solid support by passive absorption.

Embodiment E7 is the solid support of any one of embodiments E1 to E6, wherein the first ligand comprises a Zika NS1 polypeptide or antibody binding fragment thereof.

Embodiment E8 is the solid support of embodiment E7, wherein the Zika NS1 polypeptide or antibody binding fragment thereof comprises a sequence having at least 80%, at least 85%, at least 90%, at least 91%, at least 92%, at least 93%, at least 94%, at least 95%, at least 96%, at least 97%, at least 98%, at least 99%, or 100% amino acid sequence identity to a sequence selected from SEQ ID NOs: 9-16 and 18.

Embodiment E9 is the solid support of any one of embodiments E1 to E8, wherein the second ligand comprises a Zika E-protein polypeptide or antibody binding fragment thereof.

Embodiment E10 is the solid support of embodiment E9, wherein the Zika E-protein polypeptide or antibody binding fragment thereof comprises a sequence having at least 80%, at least 85%, at least 90%, at least 91%, at least 92%, at least 93%, at least 94%, at least 95%, at least 96%, at least 97%, at least 98%, at least 99%, or 100% amino acid sequence identity to a sequence selected from SEQ ID NOs: 1-8 and 17.

Embodiment F1 is a solution comprising the solid support of any one of embodiments E1 to E10.

Embodiment G1 is a solution comprising a first ligand, a second ligand, and a plurality of beads, wherein the first ligand binds to a variable region of a Zika Non-structural Protein 1 (NS1) antibody (anti-NS1 antibody) and the second ligand binds to a variable region of a Zika Envelope-protein (E-protein) antibody (anti-E-protein antibody), wherein the plurality of beads attach to complexes formed by the first ligand bound to the variable region of the anti-NS1 antibody and/or to complexes formed by the second ligand bound to the variable region of the anti-E-protein antibody.

Embodiment G2 is the solution of embodiment G1, wherein the first ligand comprises a Zika NS1 polypeptide or antibody binding fragment thereof.

Embodiment G3 is the solution of embodiment G2, wherein the Zika NS1 polypeptide or antibody binding fragment thereof comprises a sequence having at least 80%, at least 85%, at least 90%, at least 91%, at least 92%, at least 93%, at least 94%, at least 95%, at least 96%, at least 97%, at least 98%, at least 99%, or 100% amino acid sequence identity to a sequence selected from SEQ ID NOs: 9-16 and 18.

Embodiment G4 is the solution of any one of embodiments G1 to G3, wherein the second ligand comprises a Zika E protein polypeptide or antibody binding fragment thereof.

Embodiment G5 is the solution of embodiment G4, wherein the Zika E-protein polypeptide or antibody binding fragment thereof comprises a sequence having at least 80%, at least 85%, at least 90%, at least 91%, at least 92%, at least 93%, at least 94%, at least 95%, at least 96%, at least 97%, at least 98%, at least 99%, or 100% amino acid sequence identity to a sequence selected from SEQ ID NOs: 1-8 and 17.

Embodiment G6 is the solution of any one of embodiments G1 to G5, wherein the first ligand and the second ligand are in a ratio of between 1:0.5 to 1:1.5.

Embodiment G7 is the solution of any one of embodiments G1 to G5, wherein the first ligand and the second ligand are in a ratio of about 1:1.

Embodiment H1 is a kit comprising the solid support or the solution of any one of embodiments E1 to G7, and a detectable label.

Embodiment H2 the kit of embodiment H1, wherein the detectable label can be detected using an assay selected from the group consisting of an enzyme-linked immunosorbent assay (ELISA), a radioimmunoassay (RIA), an immunoprecipitation assay, a radioimmunoprecipitation (RIP) assay, an electrochemiluminescence assay, a chemiluminescence assay, and a fluorescence assay.

Embodiment H3 the kit of embodiment H1 or H2, which is for use in determining the level of the anti-NS1 antibody and the level of the anti-E-protein antibody in a biological sample.

Embodiment H4 the kit of embodiment H3, wherein the biological sample is a body fluid sample selected from the group consisting of whole blood, serum, plasma, urine, saliva, seminal fluid, cerebrospinal fluid, and a combination thereof.

Embodiment H5 the kit of embodiment H4, wherein the biological sample is serum and/or plasma.

Embodiment I1 is a Zika virus hyperimmune composition prepared according to the method of embodiment B1 or B2.

Embodiment J1 is a method of treating, preventing, or reducing the risk of a Zika virus infection in a subject, comprising administering the Zika virus hyperimmune composition of embodiment I1 to the subject.

Embodiment J2 is the method of embodiment J1, wherein the administration treats, prevents or reduces the risk of symptoms associated with a Zika virus infection.

Embodiment J3 is the method of embodiment J2, wherein the symptoms associated with the Zika virus infection comprise a fever, rash, headache, joint pain, conjunctivitis, or muscle pain.

Embodiment J4 is the method of any one of embodiments J1 to J3, wherein the Zika virus hyperimmune composition is administered intravenously or intramuscularly.

The contents of all references, GenBank entries, patents and published patent applications cited throughout this application are expressly incorporated herein by reference.

### EXAMPLES

The following examples are illustrative, but not limiting, of the present disclosure. Other suitable modifications and adaptations of the variety of conditions and parameters normally encountered in the field, and which would be apparent to those skilled in the art, are within the spirit and scope of the disclosure.

### Example 1: Development of a Multiplex Method to Screen for Anti-Zika Virus Antibodies in Plasma Samples

To differentiate between anti-Zika or anti-dengue antibodies, a multiplex screening method was developed as described below.

### Coupling:

Approximately 2.5×10⁶ magnetic COOH beads were removed from the stock vial and transferred to 2.0 mL microtube protected from light. Tubes were centrifuged at 8000 g for 1 minute and the supernatant removed. Beads were then resuspended with 100 μL laboratory water, vortexed, and sonicated for approximately 20 seconds. Tubes were again centrifuged (8000 g for 1 minute) and the supernatant removed. Next, to activate the beads, 160 μL activation buffer (50 mM MES, pH 6.0) and 20 μL of EDC (1-Ethyl-3-[3-dimethylaminopropyl] carbodiimide hydrochloride) and 20 μL Sulfo-NHS (N-hydroxysulfosuccinimide) (50 mg/mL) were added to the tubes, and the tubes were incubated at room temperature for 30 minutes, vortexing every 10 minutes. After the incubation, the tubes were centrifuged (8000 g for 1 minute) and the supernatant removed. Beads were washed twice with 500 μL coupling buffer (50 mM MES, pH 5.0).

Next, to couple the antigens to the beads, 10 μg of the following antigens were added to separate tubes containing the beads: (i) Zika lysate (i.e., Zika virus that has been chemically disrupted/inactivated), (ii) Zika virus non-structural 1 protein, (iii) Zika envelope protein, and (iv) Type 2 Dengue antigen. Tubes were brought to a final volume of 1 mL with coupling buffer and incubated at room temperature for 120 minutes on an orbital shaker set to 135-145 rpm. Afterwards, the tubes were centrifuged (8000 g for 1 minute) and the supernatant removed. Beads were resuspended with 1 mL blocking buffer (PBS, 1% BSA) and incubated at room temperature for 30 minutes on an orbital shaker set to 135-145 rpm. After the incubation, the tubes were centrifuged (8000 g for 1 minute) and the supernatant removed. Beads were then washed twice with 1 mL PBS-T and resuspended in 0.6 mL storage buffer (PBS, 1% BSA, 0.05% Azide). Bead recovery was determined using a hemocytometer.

### Multiplex Assay:

Samples (including the controls) were diluted 1:200 in the assay buffer. Samples tested included: (i) plasma from 7 yellow fever vaccinates individuals, (ii) 10 normal plasma samples, (iii) 5 dengue infected individual plasma samples, and (iv) 9 Zika infected individual plasma samples. Both 50 μL of the samples (including the controls) and 50 μL of the beads (diluted to a concentration of 20,000 beads/mL) were added to the relevant wells of a non-binding flat bottom plate. The plate was then incubated at room temperature for 60 minutes on an orbital shaker set to 175 RPM and washed with PBS-T. Next, 100 μL of anti-human IgG PE-labeled antibody (diluted 1:250 in the assay buffer) was added to the wells of the assay plate. The plate was incubated for 30 minutes at room temperature on an orbital shaker set to 175 RPM. Afterwards, the plate was washed with PBS-T. The beads in each of the wells were resuspended in 100 μL PBS-T and the plate was read using a MAGPIX Luminex Multiplex instrument. Beads were excited at multiple wavelengths. The MAGPIX detects the distinct wavelengths emitted and uses them to classify the type of beads (i.e., coupled to what antigen) and determines the median fluorescent intensity (MFI) of the PE label, which is proportional to the amount of bound antibodies.

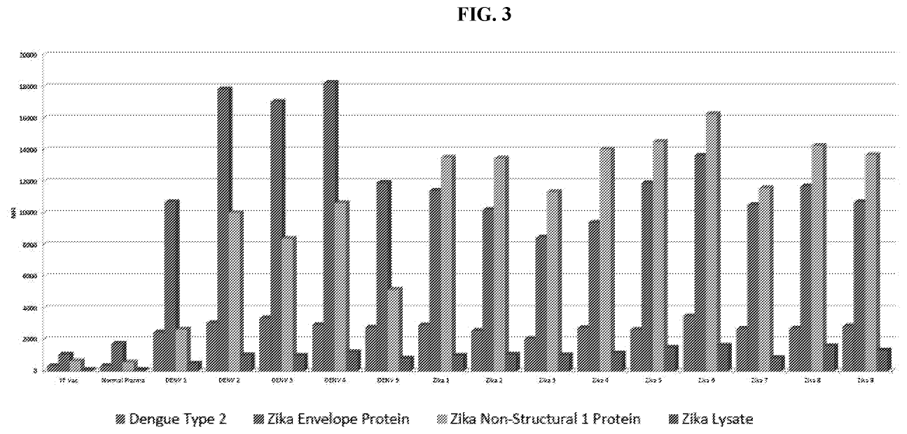

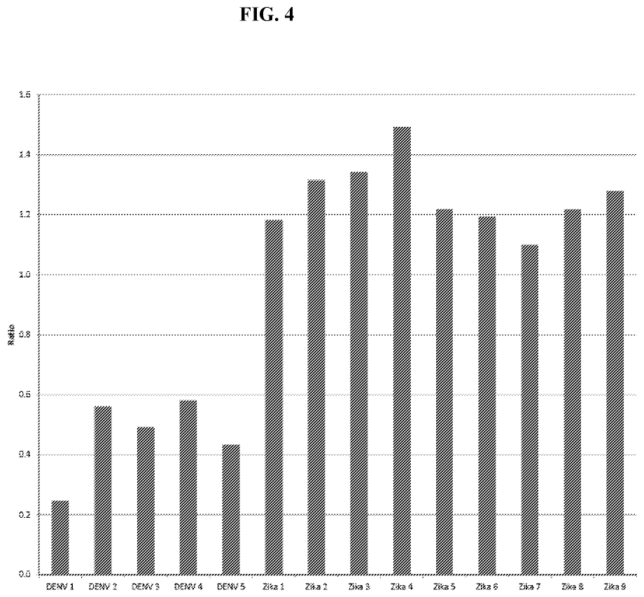

As shown in FIG. 3, the MFI signal of the Dengue Type 2 antigen (left most bar) and the Zika Lysate (right most bar) peaked at less than 4,000 MFI and 2,000 MFI, respectively, and therefore were not specific enough to differentiate between zika and dengue infections. The MFI for the E-protein (2^{nd} bar from the left) and the NS1 (3^{rd} bar from the left) were significantly different when the mean results obtained from a pool of donors were compared, but there was overlap between the individual results from Zika and Dengue infected individuals. However, a unique relationship was determined between the NS1 and E-protein signals among the Zika and Dengue infected individuals. Plasma collected from individuals exposed to the Dengue virus had higher MFI signals for E-protein in comparison to NS1. The opposite was observed for plasma samples from Zika virus infected individuals. The ratios of the NS1 MFI signal to the E-protein MFI signal for the Dengue infected individuals and the Zika infected individuals are shown in FIG. 4.

These results show that using the NS1/E-protein ratio in combination with the MFI signal, it was possible to distinguish individuals who had been exposed to the Zika virus from those who had been exposed to other flavivirus (e.g., Dengue).

### Example 2: Zika Virus Multianalyte Immunoassay

This example describes a multianalyte immunoassay for identifying individuals previously exposed to a Zika virus. Such individuals are more likely to have elevated titers of antibodies against Zika virus and are potential donors for use in preparing a Zika virus hyperimmune composition. For this assay, antibodies against the Zika virus non-structural 1 protein (NS1) (anti-NS1 antibody) and antibodies against the Zika virus envelope protein (E-protein) (anti-E-protein antibody) were detected using a MAGPIX Multiplex Reader. The Reagents/Chemicals and abbreviations used are shown in Table 1 and Table 2, respectively.

TABLE 1
Reagents/Chemicals

| Chemicals/ | | Catalog/Reagent | Storage |
|---|---|---|---|
| Reagents | Supplier | Number | Temperature |
| Zika Virus NS1 | Cedarlane/Meridian | R01636 | <−20° C. |
| | Life Science Inc. | | |
| Zika Virus | Cedarlane/Meridian | R01635 | <−20° C. |
| E-protein | Life Science Inc. | | |
| PBS w/Tween-20 | Sigma | P3536 | 2-8° C. |
| Antigen Coupled | Emergent | In-house | 2-8° C. |
| Magnetic Beads | | Preparation | (dark) |
| MAGPIX | Cederland/Luminex | MPX-PVER- | 2-8° C. |
| Performance | | K25 | |
| Verification Kit | | | |
| MAGPIX | Cederland/Luminex | MPX-CAL-K25 | 2-8° C. |
| Calibration Kit | | | |
| MAGPIX | Cederland/Luminex | MPXDF-4PK | RT |
| Drive Fluid | | | |
| PE-conjugated | Cedarlane/Jackson | 109-115-098 | 2-8° C. |
| AffiniPure Goat | Immunoresearch | | |
| Anti-Human | Labs | | |
| IgG Fcγ | | | |
| Antibody | | | |
| BSA Powder | Sigma (or | A3059 | 2-8° C. |
| | equivalent) | | |
| PBS | Sigma (or | P3813 | |
| | equivalent) | | |
| 1M HEPES | Sigma (or | 83264 | 2-8° C. |
| | equivalent) | | |
| NaCl | Fisher | S271 | RT |
| | (or equivalent) | | |
| 95% Ethanol | Emergent/Comalc | 150009/ | RT |
| | | P210EA95 | |
| 10-20% Bleach | Emergent/Univar | 150193/067096 | RT |
| 1N NaOH | Sigma (or | 1310-73-2 | RT |
| | equivalent) | | |

TABLE 2
Abbreviations

| Abbreviation | Term |
|---|---|
| BSA | Bovine Serum Albumin |
| E-Protein | Zika Virus Envelope Protein Recombinant |
| Fcγ | Fragment Crystallizable from Human Gamma Globulin |
| HEPES | (4-(2-hydroxyethyl)-1-piperazineethanesulfonic acid |
| MFI | Mean Fluorescent Intensity |
| NaCl | Sodium Chloride |
| NS1 | Zika Virus Non-Structural Protein 1 Recombinant |
| PBS-T | Phosphate Buffered Saline - 0.05% TWEEN-20 |
| PE | Phycoerythrin |
| RPM | Revolutions Per Minute |

### Controls Preparation:

Anti-Zika Positive Control: Each of Z-66, Z-128, Z-149, Z-156, and Z-91 (Antibody Systems) positive samples (or equivalent Zika positive donors) (111 μL) were combined with dilution buffer (99.4 mL, 1% BSA in PBS). Then, 90 μL aliquots, 1.5 mL aliquots, and/or 12 mL aliquots were used to make a 1:100 dilution.

Borderline Control (Anti-Dengue Positive): Each of PLA_114, PLA_116, PLA_117, and PLA_113 Dengue positive samples (or equivalent Dengue positive donors) (200 μL) were combined with the dilution buffer (99.2 mL). Then, the different aliquots were prepared as described above to make a 1:100 dilution.

Anti-Zika Negative Control: Each of ten normal anti-Zika negative plasma samples (100 μL) were combined with the dilution buffer (99 mL). Then, 1:100 dilution aliquots were prepared as described above.

### Reagent Preparation:

Coupling Procedure: Magnetic COOH beads (MAGPIX, each bead site is embedded with a unique combination of two dyes) were activated as described above in Example 1. To couple the activated beads to the relevant antigens, 20 μg of Zika Virus Envelope Protein Recombinant (Meridian Life Science Inc. Cat. No. R01635) or 20 μg of Zika Virus NS1 Protein Recombinant was added to the tubes containing the activated beads and the total volume was brought to 1 mL with coupling buffer. The beads were then incubated and washed, and the bead recovery determined as described above in Example 1.

Zika Virus NS1 Recombinant and Envelope Protein Coupled Beads: To make a 1× scale, 10 μg of either the NS1 protein or the Envelope protein were coupled to magnetic beads. To make a 2× scale, the amount of the antigen was doubled (i.e., 20 μg). The antigen coated beads were stored at 2-8° C. and protected from light until use (within 6 months).

Working Bead Dilution (20,000 beads/mL): The coupled beads were vortexed on medium speed for thirty seconds followed by thirty seconds of sonication. Then the coupled beads were added to the assay buffer to create a working bead dilution of 20,000 beads/mL. Provided below is a sample calculation:

Initial Volume=(Final Concentration×Final Volume)/Initial Concentration Equation 1:

Site 042 NS1 coupled beads (3.9×10⁶ beads/mL):

Initial Volume=[(20,000 beads/mL) (6.0 mL)]/3.9×10⁶ beads/mL=30.8 μL

Site 022 Envelope coupled beads (4.1×10⁶ beads/mL):

Initial Volume=[(20,000 beads/mL) (6.0 mL)]/4.1×10⁶ beads/mL=29.3 μL

### Procedures:

Samples were thawed at room temperature, in a 37° C. water bath, or overnight at 2-8° C. Once thawed, each of the samples were diluted in the assay buffer (150 mM NaCl, 0.1% BSA, 20 mM HEPES) to prepare two independent 1:200 dilutions for each of the samples.

Next, the antigen coated magnetic beads were vortexed and diluted in the assay buffer (20,000 beads/mL). Then, 50 μL of the diluted beads were added to each of the wells of a flat bottom nonbinding 96-well assay plates. The diluted samples (see above) (including the controls—i.e., anti-Zika positive control, borderline control, and anti-Zika negative control) were added to the relevant wells (50 μL/well). The plates were then sealed with a plate sealer and incubated in the dark at room temperature for approximately 60 minutes on an orbital shaker set to 175 RPM. After the 1 hour incubation, the plates were washed twice using a Bio-Tek 405 TS plate washer with PBS-T.

Next, PE-conjugated anti-human IgG Fcγ antibody (diluted at 1:250 in the assay buffer) were added (100 μL/well) to the relevant wells of the assay plates. The plates were sealed and incubated in the dark at room temperature for approximately 30 minutes on an orbital shaker at 175 RPM. Afterwards, the plates were washed again (twice) using the Bio-Tek 405 TS plate washer with PBS-T.

To read the plates, 100 μL of the PBS-T wash buffer was added to the wells to resuspend the beads. The plates were sealed and placed on an orbital shaker for a minimum of thirty seconds. Then, the plates were placed in the MAGPIX Multiplex reader and the fluorescence of the beads was measured using the MAGPIX XPONENT software.

### Analysis of E-Protein and NS1 MFI—Background Signal:

The average E-protein and NS1 MFI signals were calculated for all the samples (including the controls). Then, the background subtracted NS1 MFI signal (NS1 MFI—background signal) relative to that of the positive control sample was calculated using the following formula: (NS1 sample result/positive control NS1 result)×100.

For this assay, if the NS1 MFI signal was less than 20% of the positive control signal (e.g., corresponding signal observed in pooled Zika virus positive plasma samples), the sample was considered negative (i.e., not likely Zika virus related). However, if the NS1 MFI signal was greater than 20% of the positive control signal, the NS1/E-protein ratio was determined.

### Calculation of the NS1/E-Protein Ratio and Determination of Antibody Titer:

The NS1/E-protein ratio for each of the samples (including the controls) was calculated using the following formula: (NS1 MFI—background)/(E-protein MFI—background).

A sample was considered as “Anti-Zika Probable” (i.e., sample contains anti-Zika virus binding antibodies, likely from a primary Zika virus infection) if (1) the NS1 MFI signal relative to that of the Positive Control signal was greater than 20%, and (2) the NS1/E-protein ratio was greater than that of the borderline ratio (the level of anti-NS1 antibody to the level of anti-E-protein antibody measured for the borderline control sample).

To determine the titer of the anti-Zika-specific antibodies present in the Anti-Zika Probable samples, the background subtracted NS1 MFI signal relative to that of the positive control was used. If the sample signal was about 20-40% of the positive signal, the sample was considered to have low titer. If the sample signal was about 41-70% of the positive signal, the sample was considered to have medium titer. If the sample signal was greater than 70% of the positive signal, then the same was considered to have high titer. The low/med/high titers were used to rank and compare donors for use in preparing a Zika virus hyperimmune compositions.

Acceptance Criteria for this Assay:

Positive Control: NS1/E-protein ratio ≥0.8.

Blank: Blank MFI for NS1 and E-protein ≤30.

Borderline Control: Borderline NS1 MFI <Positive Control NS1 MFI.

### Example 3: Testing of Zika and Dengue Convalescent Serum for Indications of Primary or Secondary Infection and Days Post Infection Using a Multiplex Method

Human convalescent plasma samples from individuals who had dengue or Zika infections were obtained from the NIAID NIH Vaccine Research Center through BEI Resources. Samples were characterized by NIAID according to the number of days post infection and the infection type (primary or secondary) when the information was available.

Samples were tested using the multiplex method outlined in example 2 and identified as Zika positive or negative based on the binding response to Zika NS1 and envelope protein antigens. Screening results of BEI Resources serum samples are shown in Table 3.

### Results:

The multiplex method was able to positively identify Zika antibodies in 93% of samples (primary and secondary infections) as early as 13 days post infection. Early post infection samples (≤26 days) were characterized by the method as having a high non-structural 1 protein to envelope protein binding ratio. The secondary status of the infection may have had an impact on the classification of one Zika sample. It was noted that secondary Zika infections had a reduced NS1/envelope protein ratio compared to samples from known primary infections. This reduction in ratio in one secondary infection Zika sample contributed to it being classified as Negative. If plasma classification criteria was changed to require a ratio of greater than 2, it might be possible to omit samples with preexisting anti-Dengue antibodies from a prospective anti-Zika hyperimmune pool. The number of days post infection did not limit the multiplex method's ability to identify Zika antibodies in the sample as there was no strong correlation seen between the days post infection and the median fluorescence intensity signal.

Five convalescent dengue samples were tested with the multiplex method and all results were negative for Zika antibodies. This confirms the methods ability to discriminate between antibodies generated from a dengue infection caused by serotype 2, 3 or a combination thereof and a Zika infection.

TABLE 3
Screening Results of BEI Resources Serum Samples

| | | | Days | NS1/ | % of | |
|---|---|---|---|---|---|---|
| | Virus | Primary/ | Post | E-Protein | Pos | |
| Sample ID | Type | Secondary | Infection | Ratio | NS1 | Screening Result |
| NR-50919 Z | ZIKV | NS | 13 | 32.5 | 36% | Anti-Zika Probable low titer |
| NR-50921 Z | ZIKV | NS | 26 | 32.0 | 68% | Anti-Zika Probable mid titer |
| NR-50311 Z | ZIKV | NS | 31 | 1.6 | 76% | Anti-Zika Probable high titer |
| NR-50934 Z | ZIKV | NS | 37 | 7.3 | 72% | Anti-Zika Probable high titer |
| NR-50904 Z | ZIKV | NS | 62 | 1.4 | 70% | Anti-Zika Probable mid titer |
| NR-50936 Z | ZIKV | NS | 68 | 5.6 | 90% | Anti-Zika Probable high titer |
| NR-50976 Z | ZIKV | NS | 81 | 5.1 | 71% | Anti-Zika Probable high titer |
| NR-50900 Z | ZIKV | NS | 100 | 7.5 | 97% | Anti-Zika Probable high titer |
| NR-50915 Z | ZIKV | NS | 114 | 3.3 | 99% | Anti-Zika Probable high titer |
| NR-50620 Z | ZIKV | Primary | 202 | 5.7 | 108% | Anti-Zika Probable high titer |
| NR-50616 Z | ZIKV | Primary | 224 | 6.3 | 50% | Anti-Zika Probable mid titer |
| NR-50612 Z | ZIKV | Secondary | 233 | 1.4 | 93% | Anti-Zika Probable high titer |
| NR-50622 Z | ZIKV | Primary | 246 | 4.4 | 77% | Anti-Zika Probable high titer |
| NR-50618 Z | ZIKV | Secondary | 267 | 0.8 | 40% | Negative |
| NR-50226 D | DENV2 | Primary | NS | 0.3 | 2% | Negative |
| NR-50227 D | DENV2 | Primary | NS | 0.2 | 2% | Negative |
| NR-50228 D | DENV3 | Primary | NS | 1.3 | 1% | Negative |
| NR-50229 D | DENV3 | Primary | NS | 0.6 | 6% | Negative |
| NR-50233 D | DENV | Secondary | NS | 0.1 | 5% | Negative |

NS = Not specified

What is claimed is:

1. An in vitro method for identifying a donor for use in preparing a Zika virus hyperimmune composition, comprising:
determining a level of an antibody against a Zika Non-Structural protein 1 (anti-NS1 antibody) and a level of an antibody against a Zika Envelope protein (anti-E-protein antibody) in a biological sample from a potential donor;
wherein the potential donor is the donor for use in preparing a Zika virus hyperimmune composition if (i) both the anti-NS1 antibody and the anti-E-protein antibody are present in the biological sample; and (ii) the ratio of the level of the anti-NS1 antibody to the level of the anti-E-protein antibody is greater than about 0.6.

2. The method of claim 1, wherein the level of the anti-NS1 antibody in the biological sample is at least 20% of a level of the anti-NS1 antibody in a positive control sample obtained from one or more individuals previously infected with Zika virus.

3. The method of any of the previous claims, wherein the biological sample is contacted with a first ligand and a second ligand, wherein the first ligand binds to a variable region of the anti-NS1 antibody and wherein the second ligand binds to a variable region of the anti-E-protein antibody.

4. The method of claim 3, wherein the first ligand is bound to a first solid support and wherein the second ligand is bound to a second solid support.

5. The method of claim 4, wherein the first solid support is contacted with a first detectable label and the second solid support is contacted with a second detectable label, wherein the first detectable label binds to a constant region of the anti-NS1 antibody and wherein the second detectable label binds to a constant region of the anti-E-protein antibody.

6. The method of any one of claims 3 to 5, wherein the first ligand comprises a Zika NS1 polypeptide or antibody binding fragment thereof.

7. The method of claim 6, wherein the Zika NS1 polypeptide or antibody binding fragment thereof comprises a sequence having at least 80%, 85%, 90%, 91%, 92%, 93%, 94%, 95%, 96%, 97%, 98%, 99%, or 100% amino acid sequence identity to a sequence selected from SEQ ID NOs: 9-16 and 18.

8. The method of any one of claims 3 to 7, wherein the second ligand comprises a Zika E-protein polypeptide or antibody binding fragment thereof.

9. The method of claim 8, wherein the Zika E-protein polypeptide or antibody binding fragment thereof comprises a sequence having at least 80%, 85%, 90%, 91%, 92%, 93%, 94%, 95%, 96%, 97%, 98%, 99%, or 100% amino acid sequence identity to a sequence selected from SEQ ID NOs: 1-8 and 17.

10. The method of any one of claims 1 to 9, wherein the donor for use in preparing a Zika virus hyperimmune composition was not previously infected with a non-Zika flavivirus.

11. The method of any of the previous claims further comprising collecting plasma and/or serum from the donor for preparing the Zika virus hyperimmune composition.

12. The method of claim 11 further comprising preparing immunoglobulin from the plasma and/or serum collected from the donor.

13. The method of claim 11 or 12 further comprising pooling the collected plasma, collected serum, or prepared immunoglobulin for preparing the Zika virus hyperimmune composition.

14. The method of any one of claims 11 to 13 further comprising processing the pooled plasma, serum, or immunoglobulin for preparing the Zika virus hyperimmune composition.

15. A method of preparing a Zika virus hyperimmune composition, the method comprising:
(a) identifying the donor for use in preparing the Zika virus hyperimmune composition according to any one of claims 1 to 10;
(b) collecting plasma and/or serum from the donor;
(c) pooling the collected plasma and/or serum; and
(d) processing the pooled plasma and/or serum.

16. The method of claim 15, wherein the Zika virus hyperimmune composition comprises the processed plasma and/or serum of (d).

17. A method for differentiating between Zika virus antibodies and Non-Zika flavivirus antibodies in a biological sample from a subject comprising:
(a) detecting the presence or absence of antibodies against a Zika Non-Structural protein 1 (anti-NS1 antibody) and the presence or absence of antibodies against a Zika Envelope protein (anti-E-protein antibody) in a biological sample, wherein the presence of both the anti-NS1 antibodies and the anti-E-protein antibodies indicates that the sample is a flavivirus-positive biological sample;
(b) determining a level of the anti-NS1 antibody and a level of the anti-E-protein antibody in the flavivirus positive biological sample;
wherein the flavivirus positive biological sample is from a subject with Zika-virus antibodies if the level of the anti-NS1 antibody is greater than the level of the anti-E-protein antibody.

18. The method of claim 17, wherein the ratio of the level of the anti-NS1 antibody to the level of the anti-E-protein antibody is greater than about 0.6.

19. The method of claim 17, wherein the flavivirus-positive biological sample is from a subject with Non-Zika flavivirus antibodies if the level of the anti-E-protein antibody is greater than the level of the anti-NS1-protein antibody.

20. The method of any one of claims 17 to 19, wherein the biological sample is contacted with a first ligand and a second ligand, wherein the first ligand binds to a variable region of the anti-NS1 antibodies and wherein the second ligand binds to a variable region of the anti-E-protein antibodies.

21. The method of claim 20, wherein the first ligand is bound to a first solid support and wherein the second ligand is bound to a second solid support.

22. The method of claim 21, wherein the first solid support is contacted with a first detectable label and the second solid support is contacted with a second detectable label, wherein the first detectable label binds to a constant region of the anti-NS1 antibody and wherein the second detectable label binds to a constant region of the anti-E-protein antibody.

23. The method of any one of claims 20 to 22, wherein the first ligand comprises a Zika NS1 polypeptide or antibody binding fragment thereof.

24. The method of claim 23, wherein the Zika NS1 polypeptide or antibody binding fragment thereof comprises a sequence having at least 80%, 85%, 90%, 91%, 92%, 93%, 94%, 95%, 96%, 97%, 98%, 99%, or 100% amino acid sequence identity to a sequence selected from SEQ ID NOs: 9-16 and 18.

25. The method of any one of claims 20 to 24, wherein the second ligand comprises a Zika E-protein polypeptide or antibody binding fragment thereof.

26. The method of claim 25, wherein the Zika E-protein polypeptide or antibody binding fragment thereof comprises a sequence having at least 80%, 85%, 90%, 91%, 92%, 93%, 94%, 95%, 96%, 97%, 98%, 99%, or 100% amino acid sequence identity to a sequence selected from SEQ ID NOs: 1-8 and 17.

27. The method of any one of claims 15 to 26, wherein the Non-Zika flavivirus is dengue virus, Hepatitis C (HCV) virus, Yellow Fever virus, Japanese Encephalitis virus, or West Nile virus.

28. The method of any one of the previous claims, wherein the level of the anti-NS1 antibody and the level of the anti-E-protein antibody are determined by an enzyme-linked immunosorbent assay (ELISA), a radioimmunoassay (RIA), an immunoprecipitation assay, a radioimmunoprecipitation (RIP) assay, an electrochemiluminescence assay, a chemiluminescence assay, a fluorescence assay, label free—surface plasmon resonance (SPR), or gel blotting.

29. An in vitro method for detecting two target antibodies present in a biological sample comprising:
(a) contacting the biological sample with a first solid support bound to a first ligand, which binds to a variable region of a first target antibody, wherein the first target antibody is a Zika Non-structural Protein 1 (NS1) antibody (anti-NS1 antibody);
(b) contacting the biological sample with a second solid support bound to a second ligand, which binds to a variable region of a second target antibody, wherein the second target antibody is a Zika Envelope-protein (E-protein) antibody (anti-E-protein antibody); and
(c) detecting the presence or absence of the two target antibodies by detecting the binding or lack of binding of the first target antibody and the second target antibody to the first ligand and second ligand, respectively.

30. The method of claim 29, wherein the first ligand comprises a Zika NS1 polypeptide or antibody binding fragment thereof.

31. The method of claim 30, wherein the Zika NS1 polypeptide or antibody binding fragment thereof comprises a sequence having at least 80%, 85%, 90%, 91%, 92%, 93%, 94%, 95%, 96%, 97%, 98%, 99%, or 100% amino acid sequence identity to a sequence selected from SEQ ID NOs: 9-16 and 18.

32. The method of any one of claims 29 to 31, wherein the second ligand comprises a Zika E-protein polypeptide or antibody binding fragment thereof.

33. The method of claim 32, wherein the Zika E-protein polypeptide or antibody binding fragment thereof comprises a sequence having at least 80%, 85%, 90%, 91%, 92%, 93%, 94%, 95%, 96%, 97%, 98%, 99%, or 100% amino acid sequence identity to a sequence selected from SEQ ID NOs: 1-8 and 17.

34. The method of any one of claims 29 to 33, further comprising contacting the first solid support with a first detectable label and the second solid support with a second detectable label, wherein the first detectable label binds to a constant region of the first target antibody and wherein the second detectable label binds to a constant region of the second target antibody.

35. The method of any of claims 29 to 34, wherein the anti-NS1 antibody and the anti-E-protein antibody are detected by an enzyme-linked immunosorbent assay (ELISA), a radioimmunoassay (RIA), an immunoprecipitation assay, a radioimmunoprecipitation (RIP) assay, an electrochemiluminescence assay, a chemiluminescence assay, or a fluorescence assay.

36. The method of any one of claims 4 to 16 and 21 to 35, wherein the first and/or the second solid support comprises a plurality of beads, a plurality of microparticles, a multiwell plate, a slide, a test tube, a chip, a strip, a sheet, a filter, cross-linked gel supports, immobilized resins, microspheres, or a combination thereof.

37. The method of claim 36, wherein the first solid support comprises a plurality of beads and the second solid support comprises a plurality of beads, wherein the plurality of beads of the first and second solid supports are different, such that the first ligand and the second ligand are immobilized on separate beads.

38. The method of claim 36, wherein the first solid support comprises a plurality of beads and the second solid support comprises a plurality of beads, wherein the plurality of beads of the first and second solid supports are the same, such that the first ligand and the second ligand are immobilized on the same beads.

39. The method of any one of claims 4 to 16 and 21 to 38, wherein the first ligand and the second ligand are covalently coupled to the first and/or second solid support.

40. The method of any one of claims 4 to 16 and 21 to 38, wherein the first ligand and the second ligand are coupled to the first and/or second solid support by passive absorption.

41. The method of any of the previous claims, wherein the biological sample is a body fluid sample selected from the group consisting of whole blood, serum, plasma, urine, saliva, seminal fluid, cerebrospinal fluid, and a combination thereof.

42. The method of claim 41, wherein the biological sample is serum or plasma.

43. A solid support comprising a first ligand and a second ligand, wherein the first ligand binds to a variable region of a Zika Non-structural Protein 1 (NS1) antibody (anti-NS1 antibody) and the second ligand binds to a variable region of a Zika Envelope-protein antibody (E-protein) (anti-E-protein antibody), wherein the first ligand and the second ligand are immobilized on the solid support.

44. The solid support of claim 43, which comprises a plurality of beads, a plurality of microparticles, a multiwell plate, a slide, a test tube, a chip, a strip, a sheet, a filter, cross-linked gel supports, immobilized resins, microspheres, or a combination thereof.

45. The solid support of claim 43, which comprises a plurality of beads, wherein the first ligand and the second ligand are immobilized on separate beads.

46. The solid support of claim 43, which comprises a plurality of beads, wherein the first ligand and the second ligand are immobilized on the same beads.

47. The solid support of any one of claims 43 to 46, wherein the first ligand and the second ligand are covalently coupled to the solid support.

48. The solid support of any one of claims 43 to 46, wherein the first ligand and the second ligand are coupled to the solid support by passive absorption.

49. The solid support of any one of claims 43 to 48, wherein the first ligand comprises a Zika NS1 polypeptide or antibody binding fragment thereof.

50. The solid support of claim 49, wherein the Zika NS1 polypeptide or antibody binding fragment thereof comprises a sequence having at least 80%, 85%, 90%, 91%, 92%, 93%, 94%, 95%, 96%, 97%, 98%, 99%, or 100% amino acid sequence identity to a sequence selected from SEQ ID NOs: 9-16 and 18.

51. The solid support of any one of claims 43 to 50, wherein the second ligand comprises a Zika E-protein polypeptide or antibody binding fragment thereof.

52. The solid support of claim 51, wherein the Zika E-protein polypeptide or antibody binding fragment thereof comprises a sequence having at least 80%, 85%, 90%, 91%, 92%, 93%, 94%, 95%, 96%, 97%, 98%, 99%, or 100% amino acid sequence identity to a sequence selected from SEQ ID NOs: 1-8 and 17.

53. A solution comprising the solid support of any one of claims 43 to 52.

54. A solution comprising a first ligand, a second ligand, and a plurality of beads, wherein the first ligand binds to a variable region of a Zika Non-structural Protein 1 (NS1) antibody (anti-NS1 antibody) and the second ligand binds to a variable region of a Zika Envelope-protein (E-protein) antibody (anti-E-protein antibody), wherein the plurality of beads attach to complexes formed by the first ligand bound to the variable region of the anti-NS1 antibody and/or to complexes formed by the second ligand bound to the variable region of the anti-E-protein antibody.

55. The solution of claim 54, wherein the first ligand comprises a Zika NS1 polypeptide or antibody binding fragment thereof.

56. The solution of claim 55, wherein the Zika NS1 polypeptide or antibody binding fragment thereof comprises a sequence having at least 80%, 85%, 90%, 91%, 92%, 93%, 94%, 95%, 96%, 97%, 98%, 99%, or 100% amino acid sequence identity to a sequence selected from SEQ ID NOs: 9-16 and 18.

57. The solution of any one of claims 54 to 56, wherein the second ligand comprises a Zika E protein polypeptide or antibody binding fragment thereof.

58. The solution of claim 57, wherein the Zika E-protein polypeptide or antibody binding fragment thereof comprises a sequence having at least 80%, 85%, 90%, 91%, 92%, 93%, 94%, 95%, 96%, 97%, 98%, 99%, or 100% amino acid sequence identity to a sequence selected from SEQ ID NOs: 1-8 and 17.

59. The solution of any one of claims 54 to 58, wherein the first ligand and the second ligand are in a ratio of between 1:0.5 to 1:1.5.

60. The solution of any one of claims 54 to 58, wherein the first ligand and the second ligand are in a ratio of about 1:1.

61. A kit comprising the solid support or the solution of any one claims 43 to 60, and a detectable label.

62. The kit of claim 61, wherein the detectable label can be detected using an assay selected from the group consisting of an enzyme-linked immunosorbent assay (ELISA), a radioimmunoassay (RIA), an immunoprecipitation assay, a radioimmunoprecipitation (RIP) assay, an electrochemiluminescence assay, a chemiluminescence assay, and a fluorescence assay.

63. The kit of claim 61 or 62, which is for use in determining the level of the anti-NS1 antibody and the level of the anti-E-protein antibody in a biological sample.

64. The kit of claim 63, wherein the biological sample is a body fluid sample selected from the group consisting of whole blood, serum, plasma, urine, saliva, seminal fluid, cerebrospinal fluid, and a combination thereof.

65. The kit of claim 64, wherein the biological sample is serum and/or plasma.

66. A Zika virus hyperimmune composition prepared according to the method of claim 15 or 16.

67. A method of treating, preventing, or reducing the risk of a Zika virus infection in a subject, comprising administering the Zika virus hyperimmune composition of claim 66 to the subject.

68. The method of claim 67, wherein the administration treats, prevents or reduces the risk of symptoms associated with a Zika virus infection.

69. The method of claim 68, wherein the symptoms associated with the Zika virus infection comprise a fever, rash, headache, joint pain, conjunctivitis, or muscle pain.

70. The method of any one of claims 67 to 69, wherein the Zika virus hyperimmune composition is administered intravenously or intramuscularly.
